(19) United States
(12) Patent Application Publication
(10) Pub. No.: US 2026/0140060 A1
(43) Pub. Date: May 21, 2026

(54) COLORIMETRIC DETECTION OF A VIRAL INFECTION

(71) Applicant: Ramot at Tel-Aviv University Ltd., Tel-Aviv (IL)

(72) Inventors: Fernando PATOLSKY, Tel-Aviv (IL); Ella BORBERG, Tel-Aviv (IL)

(73) Assignee: Ramot at Tel-Aviv University Ltd., Tel-Aviv (IL)

(21) Appl. No.: 19/458,842

(22) Filed: Jan. 25, 2026

Related U.S. Application Data
(63) Continuation of application No. PCT/IL2024/050733, filed on Jul. 24, 2024.
(60) Provisional application No. 63/528,448, filed on Jul. 24, 2023.

(51) Int. Cl. G01N 21/80; G01N 33/68

(52) U.S. Cl. CPC G01N 21/80; G01N 33/6893; G01N 2333/165; G01N 2333/9513

(57) ABSTRACT

Methods, compositions, systems, matrices, and kits for determining a presence, type and/or amount of a viral biomarker in a sample by contacting the sample with one or more agents that selectively reacts with the biomarker and with a colorimetric indicator that exhibits a colorimetric change as a result of a reaction between the agent and a viral biomarker, are provided.

### RELATED APPLICATIONS

This application is a Continuation of PCT Patent Application No. PCT/IL2024/050733 having International filing date of Jul. 24, 2024 which claims the benefit of priority of U.S. Provisional Patent Application No. 63/528,448 filed on Jul. 24, 2023. The contents of the above applications are all incorporated by reference as if fully set herein in their entirety.

### SEQUENCE LISTING STATEMENT

The XML file, entitled 105761SequenceListing.xml, created on Nov. 21, 2025, comprising 27,647 bytes, submitted concurrently with the filing of this application is incorporated herein by reference.

### FIELD AND BACKGROUND OF THE INVENTION

The present invention, in some embodiments thereof, relates to biochemical detection, and more particularly, but not exclusively, to novel systems and methods for colorimetric detecting a presence and/or amount of a virus in a sample, including, but not limited to, detecting a presence and/or amount of a coronavirus such as SARS-COV-2 in a sample.

The coronavirus disease 2019 (COVID-19) is caused by the novel severe acute respiratory syndrome coronavirus 2 (SARS-COV-2). As a coronavirus, SARS-COV-2 is an enveloped positive-sense single-stranded ribonucleic acid (RNA) virus comprised of four structural proteins: spike, envelope, membrane and nucleocapsid. After viral RNA is released into host cytosol, viral replicase polyproteins are translated from replicase genes in the RNA open reading frames. Then, host and viral proteases cleave these polyproteins into individual non-structural proteins. One of the main viral proteases is 3-chymotrypsin-like protease (3CL^{pro}; SEQ ID NO: 1) which forms the RNA polymerase [Harrison et al., Trends in Immunology 2020, 41 (12), 1100-1115; Chen et al., F1000Res 2020, 9, 129; Perlman and Netland, Nat Rev Microbiol 2009, 7 (6), 439-450].

The SARS-CoV-2 pandemic has led to high worldwide mortality, with significant social and financial effects [Kissler et al., Science 2020, 368 (6493), 860-868]. Since asymptomatic individuals cause a large portion of SARS-CoV-2 transmission [Oran and Topol, Ann Intern Med 2020, 173 (5), 362-367; Long et al., Nat Med 2020, 26 (8), 1200-1204; and Johansson et al., JAMA Netw Open 2021, 4(1), e2035057], actions taken to curb the COVID-19 pandemic rely on high-quality testing procedures aimed at detecting specific viral molecules in infected carriers [WHO Director-General's opening remarks at the media briefing on COVID-19—16 Mar. 2020].

Reverse transcription-polymerase chain reaction (RT-PCR) is one of the primary methods for detection of viruses such as SARS-CoV-2. Although RT-PCR has high sensitivity and uses non-invasive sampling, the required sample processing is intricate, and the time-consuming test is done by specialized lab personnel using high-end equipment [Ferretti et al., Science 2020, 368 (6491)].

Immunoassay approaches like enzyme-linked immunoassays (ELISA), which work based on antigen-antibody interactions, are highly sensitive and much quicker than PCR. However, immunoassays require specific and high-affinity antibodies (and sometimes expensive recombinant and conjugated antibodies), especially in the case of complex investigations, which has limited their application in routine point-of-care procedures. For solving this problem, low-cost analogs of antibodies have gained much attention in experimental studies. Multiple antibody tests have been developed to detect SARS-CoV-2, including lateral flow immunoassay (LFIA), chemiluminescence enzyme immunoassay (CLIA), and fluorescence enzyme-linked immunoassay (FIA). The majority of these assays use spike or nucleocapsid proteins of SARS-CoV-2 [Amanat et al., Nat Med 2020, 26, 1033; and Bryant et al. Sci Immunol. 2020, 5, 47, eabc6347] to detect immunoglobulin G (IgG) and/or immunoglobulin M (IgM) antibodies produced by the host immune system against the virus. The reported methods are relatively fast (several minutes), and many are compatible with POC approaches. However, the most applicable test for POC approaches, LFIA, reportedly has the lowest performance [Vengesai et al., Systematic Reviews 2021, 10, 155], and these quantitative and qualitative assays detect exposure to SARS-CoV-2 by antibody responses rather than active infection. This can aid in the identification of factors that correlate with effective immunity to SARS-CoV-2 [Martinez-Fleta et al., SARS-Cov-2 Cysteine-like Protease (Mpro) Is Immunogenic and Can Be Detected in Serum and Saliva of COVID-19-Seropositive Individuals, Infectious Diseases (Except HIV/AIDS), 2020], but then again is less suitable for diagnosing infectious individuals [P. O. of the E. Union, “C/2020/2391, Communication from the Commission Guidelines on COVID-19 in vitro diagnostic tests and their performance 2020/C 122 I/01”, Official Journal of the European Union, 2020, C-122-I, 1-7].

Recently, paper-based biosensor platforms were developed as simple, rapid, portable and low cost platforms, making them highly suitable for point-of-care (POC) mass screening [see, for example, Antiochia, Biosensors 2021, 11 (4), 110 and Anfossi et al., Biosensors, 2019, 9(1), 2].

One of the leading approaches for paper-based biosensors is lateral flow immunoassay (LFIA). A large number of LFIA-based serological tests for SARS-CoV-2 antibody detection approved by the United States food and drug administration and entered the market in less than one year since the start of the pandemic [Nguyen et al., Biosensors and Bioelectronics 2020, 152, 112015]. Most of these assays detect immunoglobulin G (IgG) and/or immunoglobulin M (IgM) antibodies produced against the virus by the host immune system by using spike or nucleocapsid proteins of SARS-CoV-2 [Amanat et al. Nat Med 2020, 26 (7), 1033-1036; and Bryant et al., Sci Immunol. 2020, 5(47), eabc6347].

A critical issue of LFIA-based serological tests is related to the cross-reactivity of SARS-CoV-2 with low-pathogenic human coronaviruses. The immune system has been shown to target regions with high sequence similarities between seasonal coronaviruses and SARS-CoV-2 [Ma et al., The Lancet Microbe 2020, 1 (4), e151], which can generate false positive results, lowering their overall performance [Vengesai et al., Systematic Reviews 2021, 10 (1), 155]. Furthermore, these assays detect exposure to SARS-CoV-2 by immune responses rather than active infection, correlating with effective immunity to SARS-CoV-2 [Martinez-Fleta et al., J Immunol. 2020, 205(11), 3130-3140], but is less suitable for diagnosing infectious individuals [Communication from the Commission Guidelines on COVID-19 in vitro diagnostic tests and their performance 2020/C 122 I/01, published by the Publications Office of the European Union, 2020].

SARS-CoV-2 proteolytic enzymes (main protease (M^{pro}), 3 chymotrypsin-like proteinase (3CL^{pro}; SEQ ID NO: 1), non-structural protein 5 (Nsp5)) perform the cleavage of SARS-CoV-2 polyproteins pp1a and pp1ab, which is an essential step in its replication [Anirban et al., Biochimica et Biophysica Acta (BBA)—Biomembranes 2018, 1860 (2), 335-346; and Chan et al. Chem Sci. 2021, 12(41), 13686-13703]. Therefore, the presence of these enzymes indicate an active viral proliferation, i.e., active infection (Harrison et al., 2020, supra). Consequently, past and current coronaviruses treatment studies targeted 3CL^{pro} [Zhu et al. ACS Pharmacol. Transl. Sci. 2020; Morse et al. 2020, LID—10.26434/Chemrxiv.11728983.V1; and Zhang et al., Science 2020, 368 (6489), 409-412].

3CL^{pro} (SEQ ID NO: 1) is a viral proteolytic enzyme that belongs to the cysteine protease class and acts as a catalyst for peptide bond hydrolysis of viral polyproteins [Jin et al. Nature 2020, 582 (7811), 289-293; Rawlings et al. Nucleic Acids Research 2014, 42 (D1), D503-D509]

Since 3CL^{pro} (SEQ ID NO: 1) is a non-structural protein, it is not exposed in the viral particle; therefore, it is not prone to linger in host fluids as do viral envelope fragments. Moreover, since 3CL^{pro} (SEQ ID NO: 1) carries out a critical function in viral replication, its activity is essential for the viral life cycle; thus, its presence is indicative of an active infection [Harrison et al. 2020 supra]. As a critical part of viral proliferation, meaning active infection, 3CL^{pro} (SEQ ID NO: 1) has been extensively studied in coronaviruses, past and current, as a target for treatment [Zhu et al., ACS Pharmacol. Transl. Sci. 2020; Morse et al. Chembiochem. 2020, 21, 5, 730-738.; and Zhang et al., Science 2020, 368, 409].

SARS-CoV-2 proteins are expressed as a single polypeptide chain that is cleaved in eleven specific sites [Ghosh et al. Biochim Biophys Acta Biomembr. 2018, 1860, 2, 335-346]. 3CL^{pro} (SEQ ID NO: 1) cleaves at specific sites of amino acid sequences, usually in the LQ*S pattern, S could be replaced with either A or G (cleaving site is marked with *) [Zhang et al., 2020, supra; and Senger, et al., Mem. Inst. Oswaldo Cruz 2020, 115]. The 3CL^{pro} catalytic site holds a catalytic dyad of C—H. The hydrolysis is catalyzed in a well-known nucleophilic reaction. First, C thiol is deprotonated by H residue, causing a nucleophilic attack of the substrates carbonyl carbon by the anionic sulfur, followed by the N-terminus of the substrate being protonated by the H residue of the catalytic site and detaching from the substrate. The C-terminus of the substrate forms thioester intermediate with C residue, which is then hydrolyzed to produce a carboxylic acid and regenerate the catalytic site. The carboxylic acid product may cause an in-vitro pH drop in a non-buffered medium [Wang et al., ACS Catal. 2020, 10, 5871; and Huang et al., Biochemistry 2004, 43, 4568].

Proteases have been recognized as essential biomarkers in many conditions, including cancer [Edwards and Murphy, Nature 1998, 394, 527], Alzheimer's [Cataldo and Nixon, PNAS 1990, 87, 3861], AIDS [Andrew et al., Current Topics in Medicinal Chemistry 2005, 5, 1589], and inflammation [Funovics et al., Anal Bioanal Chem 2003, 377, 956], and hence studies aimed targeting proteases as a target of drugs and as a diagnostic tool have been extensively conducted [B. Turk, Nat Rev Drug Discov 2006, 5, 785].

Protease detection assays could be grouped into affinity and activity assays. Since affinity assays detect protease regardless of activity, activity assays are more applicable for functional protease detection. Activity assays include colorimetric [Zhou, et al., Analyst 2014, 139, 1178], mass spectrometry-based [Hu et al., Anal. Chem. 2015, 87, 4409], and fluorescence resonance energy transfer assays [Liu et al., Biochemical and Biophysical Research Communications 2005, 333, 194]. These can achieve low detection limits (at the pM range) but cannot be applied in multiplexed sensing platforms since only a few probes can generate different signals.

More recently, nanomaterials such as noble metal nanoparticles [Kim et al., Anal. Chem. 2014, 86, 3825], quantum dots [Wu et al., Anal. Chem. 2014, 86, 10078], and graphene oxide [Jin et al., ACS Nano 2012, 6, 4864]have been introduced in protease assays with impressive detection limits and more multiplexing capabilities. However, these are prone to limitations in the stability of the reporter molecules.

An additional group of assays, in which the substrate is immobilized on the array's surface, includes electrochemical [Cao et al., Biosensors and Bioelectronics 2013, 45, 1], surface-enhanced Raman scattering [Chen et al., Nanoscale 2013, 5, 5905], and surface plasmon resonance assays [Tripathi et al., International Journal of Biological Macromolecules 2020, 164, 2622]. These provide a platform for proteases detection that could be easily multiplexed. Nonetheless, the sensitivity of these assays tends to be lower due to the substrate immobilization onto the detection surface, causing only proteases near surfaces to elicit a signal.

Some of the present inventors have previously designed and successfully practiced methods and systems for electrochemically detecting SARS-CoV-2 in a saliva sample, while targeting the proteolytic activity of 3CL^{pro} (SEQ ID NO: 1) as a biomarker of an active infection [see, for example, Borberg et al. Nat Commun. 2022, 13 (1), 6375; and PCT/IL2023/050032, published as WO 2023/131961].

Additional background art includes Pinheiro et al., ACS Omega 2021, 6, 44, 29268-29290; Hoffman et al., J. Med. Chem. 2020, 63 (21), 12725-12747; and Yakoh et al., Biosensors and Bioelectronics 2021, 176, 15, 112912.

### SUMMARY OF THE INVENTION

According to an aspect of some embodiments of the present invention there is provided a method of determining a presence and/or amount of a viral biomarker in a sample, the method comprising: contacting the sample with an agent that selectively reacts with the viral biomarker and with a colorimetric indicator, wherein the agent that selectively reacts with the viral biomarker and the colorimetric indicator are selected such that a reaction of the agent with the viral biomarker results in a colorimetric change in the colorimetric indicator, the colorimetric change being indicative of the presence and/or amount of the viral biomarker in the sample.

According to some of any of the embodiments described herein, the viral biomarker is a proteolytic enzyme.

According to some of any of the embodiments described herein, the agent that selectively reacts with the viral biomarker is a peptide substrate that the viral biomarker specifically reacts with.

According to some of any of the embodiments described herein, the agent that selectively reacts with the viral biomarker is not an antibody specific to the proteolytic enzyme.

According to some of any of the embodiments described herein, the viral biomarker is a SARS-CoV-2-specific proteolytic enzyme (e.g., 3CL^{pro}, for example, having SEQ ID NO:1, or a homolog or analog thereof).

According to some of any of the embodiments described herein, the agent that selectively reacts with the viral biomarker is a peptide substrate that the SARS-CoV-2-specific proteolytic enzyme (e.g., 3CL^{pro}) specifically cleaves.

According to some of any of the embodiments described herein, the peptide substrate is selected from a peptide having SEQ ID NO: 2 and 10-30.

According to some of any of the embodiments described herein, the colorimetric indicator is or comprises a solid matrix and the agent is associated with, or is absorbed in and/or on, the solid matrix.

According to some of any of the embodiments described herein, the contacting comprises contacting an aqueous solution that comprises the agent that selectively reacts with the viral biomarker, the colorimetric indicator; and the sample.

According to some of any of the embodiments described herein, the reaction of the agent with the viral biomarker results in a pH change and wherein the colorimetric indicator is a pH indicator.

According to some of any of the embodiments described herein, the pH indicator comprises a pH paper.

According to some of any of the embodiments described herein, the method comprises contacting the sample with the pH paper, to thereby absorb the sample to the pH paper; and contacting the pH paper having the sample absorbed thereto with the aqueous solution that comprises the agent that selectively reacts with the viral biomarker, wherein a colorimetric change in the pH paper is indicative of a presence of the viral biomarker in the sample.

According to some of any of the embodiments described herein, the method comprises contacting the pH paper with the agent that selectively reacts with the viral biomarker, to thereby absorb the agent to the pH paper; and contacting the pH paper having the agent absorbed thereto with the sample, wherein a colorimetric change in the pH paper is indicative of a presence of the viral biomarker in the sample.

According to some of any of the embodiments described herein, the method further comprises contacting the pH paper with an aqueous solution.

According to some of any of the embodiments described herein, a pH of the aqueous solution decreases upon the reaction.

According to some of any of the embodiments described herein, the aqueous solution has a pH higher by at least 0.3 pH unit, or by at least 0.5 pH unit, or by at least one pH unit, from a pH of the aqueous solution upon the reaction between the agent and a pre-determined threshold amount of the viral biomarker in the sample.

According to some of any of the embodiments described herein, the aqueous solution is characterized by an initial pH in the range of from 7.5 to 14, or from 7.5 to 11, or from 8 to 11, or from 8 to 9, or is 8.5.

According to some of any of the embodiments described herein, the method further comprises determining a level of the colorimetric change, the level being indicative of an amount of the viral biomarker in the sample.

According to some of any of the embodiments described herein, the determining is performed within a time period of from 0.5 to 5 minutes from the contacting.

According to some of any of the embodiments described herein, the determining the level of the colorimetric change comprises converting a color that forms upon the colorimetric change to a RGB signal.

According to some of any of the embodiments described herein, the RGB signal is a B/G signal.

According to some of any of the embodiments described herein, the determining the level of the colorimetric change is performed based on a look-up table that correlates between an amount of the viral biomarker and the color that forms upon the colorimetric change.

According to some of any of the embodiments described herein, the sample is a biological sample collected from a subject, the method being for determining a presence and/or level of a viral infection in the subject.

According to some of any of the embodiments described herein, the method is for determining a presence and/or level of a viral infection in a subject, wherein the biological sample is collected from the subject, and the viral biomarker is indicative of a presence of the viral infection.

According to some of any of the embodiments described herein, the biological sample is a saliva sample of a subject.

According to some of any of the embodiments described herein, a concentration of the viral biomarker in the sample is in a range of from 0.1 μg ml⁻¹ to 10 mg ml⁻¹, or from 0.12 μg ml⁻¹ to 2 mg ml⁻¹.

According to an aspect of some embodiments of the present invention there is provided a kit comprising an agent that selectively reacts with a viral biomarker, and a colorimetric indicator which is such that exhibits a colorimetric change as a result of a reaction between the agent and the viral biomarker.

According to some of any of the embodiments described herein, the kit comprises an aqueous solution of the agent that selectively reacts with the viral biomarker and the colorimetric indicator, wherein the aqueous solution and the colorimetric indicator are individually packaged in the kit.

According to some of any of the embodiments described herein, the kit comprises an aqueous solution that comprises the agent that selectively reacts with the viral biomarker and an aqueous solution that comprises the colorimetric indicator.

According to some of any of the embodiments described herein, the aqueous solution that comprises the agent that selectively reacts with the viral biomarker further comprises the colorimetric indicator.

According to some of any of the embodiments described herein, the aqueous solution that comprises the agent that selectively reacts with the viral biomarker and the aqueous solution that comprises the colorimetric indicator are individually packaged within the kit.

According to some of any of the embodiments described herein, the colorimetric indicator is or comprises a solid matrix.

According to some of any of the embodiments described herein, the agent is absorbed to the solid matrix.

According to some of any of the embodiments described herein, the reaction between the agent and the viral biomarker results in a pH change and the colorimetric indicator is a pH paper.

According to some of any of the embodiments described herein, the agent is absorbed to the pH meter, the kit further comprising an aqueous solution.

According to some of any of the embodiments described herein, the aqueous solution has a pH higher by at least 0.3 pH unit, or by at least 0.5 pH unit, or by at least one pH unit, from a pH of the solution upon the reaction between the agent and a pre-determined threshold amount of the viral biomarker in the sample.

According to some of any of the embodiments described herein, the aqueous solution is characterized by an initial pH in the range of from 7.5 to 14, or from 7.5 to 11, or from 8 to 11, or from 8 to 9, or is 8.5.

According to some of any of the embodiments described herein, the kit further comprises means or a device for collecting a biological sample from a subject.

According to some of any of the embodiments described herein, the colorimetric agent is mounted on or forms a part of the means or device.

According to some of any of the embodiments described herein, the viral biomarker is a proteolytic enzyme.

According to some of any of the embodiments described herein, the agent that selectively reacts with the viral biomarker is a peptide substrate that the viral biomarker specifically reacts with.

According to some of any of the embodiments described herein, the agent that selectively reacts with the viral biomarker is not an antibody specific to the proteolytic enzyme.

According to some of any of the embodiments described herein, the kit is devoid of an antibody.

According to some of any of the embodiments described herein, the viral biomarker is a SARS-CoV-2-specific proteolytic enzyme.

According to some of any of the embodiments described herein, the agent that selectively reacts with the viral biomarker is a peptide substrate that the SARS-CoV-2-specific proteolytic enzyme specifically cleaves.

According to some of any of the embodiments described herein, the peptide substrate that the SARS-CoV-2-specific proteolytic enzyme specifically cleaves is selected from a peptide having an amino acid sequence SEQ ID NOs: 2 and 10-30.

According to some of any of the embodiments described herein, the kit is for use in determining a presence and/or amount of a viral biomarker in a sample.

According to some of any of the embodiments described herein, the sample is a biological sample collected from a subject, the kit being for use in determining a presence and/or level of a viral infection in a subject.

According to an aspect of some embodiments of the present invention there is provided a composition or a composition-of-matter or an article-of-manufacturing comprising a colorimetric indicator which is or comprises a solid matrix and an agent that selectively reacts with a viral biomarker associated with the solid matrix. An exemplary such an article-of-manufacturing is a test strip or a testing strip as described herein in any of the respective embodiments.

According to some of any of the embodiments described herein, the agent is absorbed to the solid matrix.

According to some of any of the embodiments described herein, the reaction of the agent with the viral biomarker results in a pH change and wherein the colorimetric indicator is a pH indicator.

According to some of any of the embodiments described herein, the pH indicator comprises a pH paper.

According to some of any of the embodiments described herein, the viral biomarker is a proteolytic enzyme.

According to some of any of the embodiments described herein, the agent that selectively reacts with the viral biomarker is not an antibody specific to the proteolytic enzyme.

According to some of any of the embodiments described herein, the agent that selectively reacts with the viral biomarker is a peptide substrate that the viral biomarker specifically reacts with.

According to some of any of the embodiments described herein, the agent that selectively reacts with the viral biomarker is not an antibody specific to the proteolytic enzyme.

According to some of any of the embodiments described herein, the viral biomarker is a SARS-CoV-2-specific proteolytic enzyme.

According to some of any of the embodiments described herein, the agent that selectively reacts with the viral biomarker is a peptide substrate that the SARS-CoV-2-specific proteolytic enzyme specifically cleaves.

According to some of any of the embodiments described herein, the peptide substrate that the SARS-CoV-2-specific proteolytic enzyme specifically cleaves is selected from a peptide having SEQ ID NO: 2 and 10-30.

According to some of any of the embodiments described herein, the composition is capable of determining a presence and/or amount of the viral biomarker in a sample upon storage for at least 3 days, or at least one week, or at least two weeks, or at least a month.

According to an aspect of some embodiments of the present invention there is provided a composition comprising an aqueous solution and an agent that selectively reacts with a viral biomarker, the aqueous solution featuring a pH that is higher by at least 0.3 pH unit, or by at least 0.5 pH unit, or by at least one pH unit, than a pH of the solution upon the reaction between the agent and a pre-determined threshold amount of the viral biomarker in the sample.

According to some of any of the embodiments described herein, the viral biomarker is a proteolytic enzyme.

According to some of any of the embodiments described herein, the agent that selectively reacts with the viral biomarker is not an antibody specific to the proteolytic enzyme.

According to some of any of the embodiments described herein, the agent that selectively reacts with the viral biomarker is a peptide substrate that the viral biomarker specifically reacts with.

According to some of any of the embodiments described herein, the agent that selectively reacts with the viral biomarker is not an antibody specific to the proteolytic enzyme.

According to some of any of the embodiments described herein, the viral biomarker is a SARS-CoV-2-specific proteolytic enzyme.

According to some of any of the embodiments described herein, the agent that selectively reacts with the viral biomarker is a peptide substrate that the SARS-CoV-2-specific proteolytic enzyme specifically cleaves.

According to some of any of the embodiments described herein, the peptide substrate that the SARS-CoV-2-specific proteolytic enzyme specifically cleaves is selected from a peptide having SEQ ID NO: 2 and 10-30.

According to some of any of the embodiments described herein, the composition further comprises a colorimetric indicator, wherein the reaction of the agent with the viral biomarker results in a pH change, and wherein the colorimetric indicator is a pH indicator.

According to some of any of the embodiments described herein, the pH indicator comprises a pH paper.

According to an aspect of some embodiments of the present invention there is provided a colorimetric indicator-containing matrix, comprising a colorimetric indicator which is or comprises a solid matrix and at least a first agent and a second agent each associated with the solid matrix, wherein the first agent specifically reacts with a first viral biomarker and the second agent specifically reacts with a second viral biomarker.

According to some of any of the embodiments described herein, the matrix further comprises a third agent, a fourth agent, and so forth, each independently reacts specifically with a different viral biomarker.

According to some of any of the embodiments described herein, the matrix is for use in determining a presence, a type and/or amount of a viral infection in a subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a SEM image presenting the fibers of a pH indicator paper according to some embodiments of the present invention. Scale bar is 250 μm.

FIGS. 2A-B are schematic illustrations of an exemplary SARS-CoV-2 detection method, showing saliva sampling (FIG. 2A) and optical readout of the samples from FIG. 2A and analysis of colorimetric data using a cellphone app to translate RGB values into positive/negative diagnostic test results (FIG. 2B).

FIG. 3A presents photographs of representative pH-papers taken at different pH values in the range of 5.5-8.5, and the respective red (R), green (G) and blue (B) values.

FIGS. 3B-3E are inclining plots showing the pH-dependency of RGB vector (FIG. 3B; calculated using the equation: RGB vector=\sqrt{R²+G²+B²}); B²/RG value (FIG. 3C); B/G value (FIG. 3D); and B/R value (FIG. 3E), as calculated from the values of each of the samples shown in FIG. 3A.

FIG. 4A presents a table showing photographs of pH indicator papers following exposure to a basic solution (pH=8.5) containing an exemplary 3CL^{pro}-specific substrate (SEQ ID NO: 2), and to healthy saliva samples spiked with different concentrations of 3CL^{pro} (SEQ ID NO: 1) at varying initial pH values (5.5, 6.5 and 7.5).

FIGS. 4B-D present comparative plots showing the dose-dependent increase in B²/RG values, and the corresponding fitting parameters (slopes and intercepts with standard deviations, and coefficient of determination, R²), for each of the samples shown in FIG. 4A, pH=5.5 (black), 6.5 (red) and 7.5 (blue).

FIGS. 4E-G present comparative plots showing the dose-dependent increase in B²/RG (FIG. 4E), B/G (FIG. 4F) and B/R (FIG. 4G) signals, calculated by deducting the initial RGB-derived value (at a viral load of 0 μg/ml 3CL^{pro} (SEQ ID NO: 1)) from the RGB-derived value of FIG. 4A.

FIG. 5A presents a table showing photographs of exemplary pH indicator papers following exposure to a basic solution (pH=8.5) containing an exemplary 3CL^{pro}-specific substrate (SEQ ID NO: 2), and to healthy saliva samples spiked with different proteolytic enzymes: an exemplary viral biomarker according to some embodiments of the present invention, 3CL^{pro} (SEQ ID NO: 1), originating from different sources (SARS-CoV-2, SARS-CoV, and MERS-CoV); human immunodeficiency virus protease (HIV-2 Protease); and the human proteases TMPRSS2 and Chymotrypsin.

FIGS. 5B-D are bar graphs presenting B/G (FIG. 5B), B²/RG (FIG. 5C) and B/R (FIG. 5D) values measured for each of the pH indicator paper upon exposure to different proteases shown in FIG. 5A.

FIG. 6A-B present comparative plots showing the time-dependent calculated absolute pH following exposure at T=0 to 3CL proteases originating from SARS-CoV-2 (black; SEQ ID NO: 1), SARS-CoV (red; SEQ ID NO: 5), and MERS-CoV (blue; SEQ ID NO: 4). pH was measured through pyranine fluorescence, and the absolute response is calculated by: (pHᵢ^{substrate}−pHₜ^{substrate})−(pHᵢ^{control}−pHₜ^{control}) FIG. 6A presents 22 minutes kinetics and FIG. 6B presents a 5-minute close-up. The vertical dotted grey line marks T=1 minute, wherein 3CL^{pro} from SARS-CoV-2 (SEQ ID NO: 1) exhibits substantially higher absolute pH response than the absolute pH obtained from 3CL^{pro} of SARS-CoV and MERS-CoV (SEQ ID NOs: 5 and 4, respectively).

FIGS. 7A-B are bar graphs showing B/G signals (FIG. 7A) and stacked column chart showing B²/RG (violet), B/G (pink), and B/R (yellow) signals (FIG. 7B) in SARS-CoV-2 PCR-negative and PCR-positive human subjects, tested using the exemplary virus detection platform according to some of the present embodiments. The RGB-derived signals were calculated by: Signal=Value₀−Valueᵢ, wherein Value₀ is the value at a viral load of 0 μg/ml 3CL^{pro} (SEQ ID NO: 1), and Valueᵢ is the obtained value at the two-minute cutoff in which measurement is taken.

FIGS. 7C-H are bar graphs showing the correlation between PCR cycle threshold (Ct) value and B/G (FIG. 7C), B²/RG (FIG. 7E) or B/R (FIG. 7G) signals, and the effect of the initial pH of the biological sample on B/G (FIG. 7D), B²/RG (FIG. 7F) or B/R (FIG. 7H) signals, based on the data presented in FIGS. 7A-B. In FIG. 7C, the blue dotted line indicates a trend of stronger signals with lower Ct values.

FIGS. 8A-C are schematic illustrations of an exemplary at-home test strip model 100 for determining a presence of a viral infection in a subject. FIG. 8A is a side-view of the test strip 100, showing a blister 18 that comprises a basic (e.g., pH=8.5) solution 20, and two windows 12 with pH indicator papers having an embedded dried solution of the agent that selectively reacts with a viral biomarker (e.g., 3CL^{pro}-specific peptide substrate; SEQ ID NO: 2); FIGS. 8B-C are top-view schematic illustrations of a negative (FIG. 8B) and positive (FIG. 8C) results in the exemplary test strip 100.

FIGS. 8D-G are photographs of an exemplary at-home test strip model 100 as schematically presented in FIGS. 8A-C. FIGS. 8D-E are top-view (FIG. 8D) and side-view (FIG. 8E) of the two-window strip before its use, wherein both windows are yellow. In FIG. 8D, scale bar 5 mm. In FIG. 8E, the backside blister comprising the ‘solution test’ is shown. FIGS. 8F-G are top-view images of a COVID-19 negative (FIG. 8B) and COVID-19 positive (FIG. 8C) results in the test strip 100. Control window and Test window 12 are indicated for each image.

FIG. 9A presents photographs of a pH indicator paper having 3CL^{pro}-specific substrate (“substrate”) (SEQ ID NO: 2) absorbed thereto (left column), when exposed sequentially to pH=8.5 buffer solution (8 μl) (second column from the left), drying (third column from the left), to a solution containing 3CL^{pro} (“protease”; 4 μl; fourth column from the left) (SEQ ID NO: 1) and additional drying (right column), demonstrating no change in colorimetric detection of the protease upon 36-days storage of the substrate-containing pH paper.

FIG. 9B presents photographs of the same substrate-containing pH paper shown in FIG. 9A, following 36-day storage, when exposed sequentially to pH=8.5 buffer solution (8 μl) (second photograph from the left), drying (third photograph from the left), and distilled water (DW), demonstrating no color change, as opposed to exposure of the protease solution.

FIG. 9C presents photographs of a pH paper with no substrate (“Clean”), which served as control, upon 36-days storage, following sequential exposure to 3CL^{pro} (“protease”) and pH=8.5 buffer solution, and 3 minutes after exposure to the buffer solution.

FIG. 9D presents a comparative photograph of two substrate-embedded pH indicator papers, following 36-days storage, side-by-side, following exposure to 8 μL pH=8.5 buffer solution and thereafter to 4 μL 3CL^{pro} (SEQ ID NO: 1) (left); and protease-free saliva sample (right).

### DESCRIPTION OF SPECIFIC EMBODIMENTS OF THE INVENTION

The present invention, in some embodiments thereof, relates to biochemical detection, and more particularly, but not exclusively, to novel systems and methods for colorimetric detecting a presence and/or amount of a virus in a sample, including, but not limited to, detecting a presence and/or amount of a coronavirus such as SARS-CoV-2 in a sample.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Although accurate virus (e.g., SARS-CoV-2) detection is achieved by methods such as RT-PCR, these methods are unsuitable for point-of-care (POC) applications, as the detection turnover rate of these methods is exceedingly low, and expensive machinery, trained personnel, and multiple expensive and sensitive reagents are required.

The present inventors have devised and successfully practiced an ultrafast (e.g., one-minute) simple smart paper-based colorimetric approach that targets viral biomarkers and has demonstrated a successful targeting of a SARS-CoV-2-specific proteolytic enzyme, 3CL^{pro} (SEQ ID NO: 1), as an exemplary viral biomarker of active infection. The self-amplifying proteolytic activity of a viral biomarker such as 3CL^{pro} (SEQ ID NO: 1) is detected directly from biological samples such as untreated saliva samples using a peptide substrate-embedded paper-based pH-reporter platform, based on the intrinsic proteolytic selectivity of the viral biomarker (e.g., 3CL^{pro} (SEQ ID NO: 1)) against the applied peptide substrate, and without the requirement of chemically modifying the sensor's surface with specific capturing antibody units to achieve sensing selectivity.

While reducing the present invention to practice, the present inventors have designed and successfully practiced a colorimetric detection methodology for determining a presence and optionally an amount of the presence of 3CL^{pro} (SEQ ID NO: 1) in a biological sample. While this method has been exemplified herein for the detection of the presence of 3CL^{pro} (SEQ ID NO: 1) as a viral biomarker, it can be readily adopted for detecting any viral biomarker that generates a pH change as a result of a viral infection.

The present inventors have successfully proved the potential of a commercially available pH-indicator paper as a platform for the reliable and ultrafast colorimetric detection of SARS-CoV-2 directly from saliva swab samples in less than one minute, without using immunologic agents (e.g., antibodies).

Surprisingly, when comparing the absolute pH change in the presence of potentially interfering proteases, within only two minutes the tested SARS-CoV-2 3CL^{pro} (SEQ ID NO: 1) resulted in about 10 times higher absolute pH response than that of analogous 3CL^{pro}s (SARS-CoV and MERS-CoV; SEQ ID NOs: 5 and 4, respectively).

Clinical tests of saliva samples from 42 subjects showed highly accurate detection of SARS-CoV-2, with 100% sensitivity and 100% specificity, validated by PCR testing of the samples.

The presence and activity of 3CL^{pro} (SEQ ID NO: 1) in saliva samples of SARS-CoV-2-positive individuals, which was detected by a color change in a substrate-embedded pH-indicator paper according to some embodiments of the present invention, was further demonstrated in a prototype. This virus detection method and system provide a combination of remarkable accuracy, specificity, and sensitivity, ultrafast detection turnover, protocol simplicity, easy integration into mobile technology, long-life stability under ambient storage conditions, and point-of-care compatibility for SARS-CoV-2 mass screening.

Combined with exceptionally low device costs and easily scalable materials, the herein disclosed methods and devices (articles-of-manufacturing) enable a large-scale, fast, and accurate SARS-CoV-2 detection platform, thus allowing timely implementation of measures to curb pandemic progression.

Due to its universal sensing approach, the detection of other viral infections is similarly implemented through the detection of viral specific biomarkers such as viral proteases (e.g., smallpox K7L protease; human rhinovirus 3CL protease; and poliovirus 2A protease). Applying agents that selectively react with biomarkers such as viral proteases in substrate-embedded reaction zones to an array of pH-paper test windows enables the straightforward multiplexed diagnosis of multiple viral infections (e.g., SARS-CoV-2 and common flu viruses) on a single antibody-free universal pH-based sensing platform.

Embodiments of the present invention therefore relate to a novel methodology of determining a presence and/or amount of a viral biomarker in a sample, which utilizes an agent that selectively reacts with the viral biomarker and a colorimetric indicator which is such that exhibits a colorimetric change as a result of a reaction between the viral biomarker and the agent that selectively reacts therewith, and to compositions and kits that comprise the colorimetric indicator and/or the agent that selectively reacts with a viral biomarker.

According to an aspect of some embodiments of the invention, there is provided a method of determining a presence and/or amount of a viral biomarker in a sample. According to the present embodiments, the method is effected by contacting the sample (e.g., a biological sample, such as described herein) with an agent that selectively (specifically) reacts with the viral biomarker (also being referred to herein interchangeably as “agent”) and with a colorimetric indicator. In some of any of the embodiments described herein, the agent that selectively reacts with the viral biomarker and the colorimetric indicator are selected such that a reaction of this agent (i.e., that selectively reacts with the viral biomarker) with the viral biomarker results in a colorimetric change in the colorimetric indicator. In some of any of the embodiments described herein, the colorimetric change is indicative of the presence and/or amount of the viral biomarker in the sample.

According to some of any of the embodiments described herein, the sample is a biological sample. According to some of these embodiments, the method is for determining a presence and/or level (e.g., progress) of a viral infection in a subject from which a biological sample is drawn. Alternatively, a method as described herein can be used simply for determining a presence and/or amount of a viral biomarker in a sample such as a biological sample (e.g., for research purposes).

Herein and in the art, the phrase “biological sample” describes a sample derived from a biological source. It encompasses samples that are drawn, collected or otherwise obtained from a biological tissue of a subject. Non-limiting examples of biological samples include a nasal secretion sample, a saliva sample, a blood sample, a urine sample, a biological tissue biopsy, a cerebrospinal fluid sample, a sweat sample, and a buccal swab sample. Non-limiting examples of biological tissues include body fluids such as whole blood, serum, plasma, cerebrospinal fluid, urine, lymph fluids, and various external secretions of the respiratory, intestinal and genitourinary tracts, tears, saliva, milk, sweat, white blood cells, malignant tissues, amniotic fluid and chorionic villi.

Exemplary biological samples include, but are not limited to, blood (e.g., peripheral blood leukocytes, peripheral blood mononuclear cells, whole blood, cord blood), saliva, a solid tissue biopsy, cerebrospinal fluid, urine, lymph fluids, and various external secretions of the respiratory, intestinal and genitourinary tracts, synovial fluid, amniotic fluid and chorionic villi.

Biopsies include, but are not limited to, surgical biopsies including incisional or excisional biopsy, fine needle aspirates and the like, complete resections or body fluids. Methods of biopsy retrieval are well known in the art.

In some embodiments, the biological sample is of a subject suspected as having a viral infection, for example, a subject suspected as having the viral infection associated with the viral biomarker.

Herein throughout, the term “subject” includes mammals, preferably human beings at any age, which suffer from the pathology (viral infection), are suspected as suffering from the pathology or who are at risk to develop the pathology.

In some of any of the embodiments described herein, the biological sample is selected as such that can comprise the viral biomarker as described herein in any of the respective embodiments, that is, is obtained from a solid tissue or fluid where a respective viral biomarker is expected to be present in a detectable level in case of a viral infection.

In some of any of the embodiments described herein, the biological sample is drawn, collected or otherwise obtained from a subject. Non-limiting methods for obtaining the biological samples include blood sampling (e.g., venipuncture, fingerstick), saliva collection (e.g., spitting into a collection tube, using a swab), urine collection (e.g., midstream catch, catheterization), tissue biopsy (e.g., needle biopsy, surgical biopsy), and swab sampling (e.g., nasal swab, throat swab, buccal swab).

In some of any of the embodiments described herein, the biological sample is used as is in a method as described herein, as an untreated sample, without being subjected to any treatment, separation technique, etc.

In some of any of the embodiments described herein, the biological sample is a fluid sample, of a fluid tissue, for example, a saliva sample.

In some of any of the embodiments described herein, the biological sample is a solid sample, e.g., of a solid tissue. In some of these embodiments, the sample may be mixed with an aqueous solution (e.g., a buffered aqueous solution).

In some of any of the embodiments described herein, a detectable concentration of the viral biomarker in the sample can be as low as 0.01 μg ml⁻¹, although lower concentrations are also contemplated. In some of any of the embodiments described herein, a detectable concentration of the viral biomarker in the sample can be as low as 0.05 μg ml⁻¹. In some of any of the embodiments described herein, a detectable concentration of the viral biomarker in the sample can be as low as 0.1 μg ml⁻¹.

In some of any of the embodiments described herein, a detectable concentration of the viral biomarker in the sample as described herein in any of the respective embodiments is in a range of from 0.01 μg ml⁻¹ to 10 mg ml⁻¹, or from 0.1 μg ml⁻¹ to 10 mg ml⁻¹, or from 0.1 μg ml⁻¹ to 9 mg ml-1, or from 0.1 μg ml⁻¹ to 8 mg ml⁻¹, or from 0.1 μg ml⁻¹ to 7 mg ml⁻¹, or from 0.1 μg ml⁻¹ to 6 mg ml⁻¹, or from 0.1 μg ml⁻¹ to 5 mg ml⁻¹, or from 0.1 μg ml⁻¹ to 4 mg ml⁻¹, or from 0.1 μg ml⁻¹ to 3 mg ml⁻¹, or from 0.11 μg ml⁻¹ to 5 mg ml⁻¹, or from 0.11 μg ml⁻¹ to 4 mg ml⁻¹, or from 0.11 μg ml⁻¹ to 3 mg ml⁻¹, or from 0.11 μg ml⁻¹ to 2 mg ml⁻¹, or from 0.12 μg ml⁻¹ to 5 mg ml⁻¹, or from 0.12 μg ml⁻¹ to 4 mg ml⁻¹, or from 0.12 μg ml⁻¹ to 3 mg ml⁻¹, or from 0.12 μg ml⁻¹ to 2.5 mg ml⁻¹, or from 0.12 μg ml⁻¹ to 2 mg ml⁻¹, including any intermediate values and subranges therebetween.

According to some of any of the embodiments described herein, the method is for determining a presence and/or level of a viral infection in a subject.

Herein throughout, a presence and/or level of a viral infection and/or a viral biomarker is determined by a presence and/or level of a colorimetric change, such that upon contacting the agent, the colorimetric indicator, and the sample, as these are described herein in any of the respective embodiments, a detectable colorimetric change is formed if a viral biomarker is present in the sample, and is thereby indicative of a presence and/or amount of the viral biomarker in the sample and/or of a presence and/or amount of a viral infection in a subject (in case a biological sample is drawn from a subject).

According to some of any of the embodiments described herein, the biological sample is a saliva sample of a subject. In such embodiments, the viral biomarker as described herein in any of the respective embodiments is present in the saliva of an infected subject. According to some of any of the embodiments described herein, the biological sample is a saliva sample, the viral biomarker is such that is present in a saliva of a subject (having the respective viral infection) and the agent that selectively reacts with the viral biomarker is selected such that it reacts with a viral biomarker that is present in the saliva.

In some of any of the embodiments described herein, the (e.g., biological) sample is collected (e.g., from the subject, as described herein), and a presence and/or amount of the colorimetric change is indicative of a presence and/or amount of the viral biomarker in the sample and/or the viral infection in the subject. In some of these embodiments, the collected sample is used without being further processed or treated.

As used herein and in the art, the phrase “viral infection” describes a presence (i.e., the invasion) and multiplication of viruses within cells of a host organism (a subject as described herein), which typically leads to cellular damage, immune response activation, and various clinical symptoms.

Herein, the term “virus” refers to an agent that replicates only inside living cells of an organism, and encompasses agents composed solely of a nucleic acid, such as viroids. Examples of viruses include, without limitation, double strand DNA viruses, such as adenoviruses, herpesviruses (e.g., varicella zoster virus, herpes simplex virus-1 and/or herpes simplex virus-2), polyomaviruses (e.g., JC virus), and poxviruses; single strand DNA viruses, such as parvoviruses; double strand RNA viruses, such as reoviruses (e.g., epizootic hemorrhagic disease virus); (+)-single strand RNA viruses, such as coronaviruses (e.g., coronavirus HKU1, coronavirus NL63, coronavirus 229E, coronavirus OC43, Middle East respiratory syndrome coronavirus (MERS-CoV) and/or SARS-CoV), flaviviruses (e.g., hepatitis C virus and/or West Nile virus), hepeviruses (e.g., hepatitis E virus), picornaviruses (e.g., hepatitis A virus, enteroviruses and/or rhinoviruses, such as human enteroviruses and/or rhinoviruses) and togaviruses; (−)-single strand RNA viruses, such as orthomyxoviruses (e.g., influenza A virus and/or influenza B virus), filoviruses (e.g., Ebola virus), paramyxoviruses (e.g., parainfluenza virus type 1, 2, 3 and/or 4), pneumoviruses (e.g., respiratory syncytial virus and/or human metapneumovirus) and rhabdoviruses (e.g., vesicular stomatitis Indiana virus); RNA retroviruses; DNA retroviruses, such as hepadnaviruses (e.g., hepatitis B virus); satellite viruses, such as deltaviruses (e.g., hepatitis D virus); and viroids.

According to some of any of the embodiments described herein, the viral infection is associated with double strand DNA viruses, single strand DNA viruses, double strand RNA viruses, (+)-single strand RNA viruses, (−)-single strand RNA viruses, RNA retroviruses; DNA retroviruses, satellite viruses, and/or viroids.

Exemplary viruses that cause disease include, but are not limited to, those set forth in Table A hereinbelow.

TABLE A

| | Baltimore | | |
|---|---|---|---|
| Family | group | Important species | Envelopment |
| Adenoviridae | Group I | Adenovirus | non-enveloped |
| | (dsDNA) | | |
| Herpesviridae | Group I | Herpes simplex, type 1, Herpes simplex, type | enveloped |
| | (dsDNA) | 2, Varicella-zoster virus, Epstein-Barr virus, | |
| | | Human cytomegalovirus, Human herpesvirus, | |
| | | type 8 | |
| Papillomaviridae | Group I | Human papillomavirus | non-enveloped |
| | (dsDNA) | | |
| Polyomaviridae | Group I | BK virus, JC virus | non-enveloped |
| | (dsDNA) | | |
| Poxviridae | Group I | Smallpox | enveloped |
| | (dsDNA) | | |
| Hepadnaviridae | Group VII | Hepatitis B virus | enveloped |
| | (dsDNA-RT) | | |
| Parvoviridae | Group II | Parvovirus B19 | non-enveloped |
| | (ssDNA) | | |
| Astroviridae | Group IV | Human astrovirus | non-enveloped |
| | (positive- | | |
| | sense ssRNA) | | |
| Caliciviridae | Group IV | Norwalk virus | non-enveloped |
| | (positive- | | |
| | sense ssRNA) | | |
| Picornaviridae | Group IV | coxsackievirus, hepatitis A virus, poliovirus, | non-enveloped |
| | (positive- | rhinovirus | |
| | sense ssRNA) | | |
| Coronaviridae | Group IV | Severe acute respiratory syndrome virus | enveloped |
| | (positive- | | |
| | sense ssRNA) | | |
| Flaviviridae | Group IV | Hepatitis C virus, yellow fever virus, dengue | enveloped |
| | (positive- | virus, West Nile virus, TBE virus | |
| | sense ssRNA) | | |
| Togaviridae | Group IV | Rubella virus | enveloped |
| | (positive- | | |
| | sense ssRNA) | | |
| Hepeviridae | Group IV | Hepatitis E virus | non-enveloped |
| | (positive- | | |
| | sense ssRNA) | | |
| Retroviridae | Group VI | Human immunodeficiency virus (HIV) | enveloped |
| | (SSRNA-RT) | | |
| Orthomyxoviridae | Group V | Influenza virus | enveloped |
| | (negative- | | |
| | sense ssRNA) | | |
| Arenaviridae | Group V | Lassa virus | enveloped |
| | (negative- | | |
| | sense ssRNA) | | |
| Bunyaviridae | Group V | Crimean-Congo hemorrhagic fever virus, | enveloped |
| | (negative- | Hantaan virus | |
| | sense ssRNA) | | |
| Filoviridae | Group V | Ebola virus, Marburg virus | enveloped |
| | (negative- | | |
| | sense ssRNA) | | |
| Paramyxoviridae | Group V | Measles virus, Mumps virus, Parainfluenza | enveloped |
| | (negative- | virus, Respiratory syncytial virus, | |
| | sense ssRNA) | | |
| Rhabdoviridae | Group V | Rabies virus | enveloped |
| | (negative- | | |
| | sense ssRNA) | | |
| Unassigned | Group V | Hepatitis D | enveloped |
| | (negative- | | |
| | sense ssRNA) | | |
| Reoviridae | Group III | Rotavirus, Orbivirus, Coltivirus, Banna virus | non-enveloped |
| | (dsRNA) | | |

According to some of any of the embodiments described herein, the viral infection affects non-human subjects. Non-limiting examples of viruses which may cause viral infections in non-human subjects include foot-and-mouth disease (FMVI), canine parvovirus, feline leukemia virus (FeLV), lentiviruses (e.g., feline immunodeficiency virus (FIV), coronaviruses (e.g., MERS), simian immunodeficiency virus (SIV), bovine immunodeficiency virus (BIV), equine infectious anemia virus (EIAV)), avian influenza, lyssaviruses (e.g., Australian bat lyssavirus; causing rabies), African swine fever (ASF), Newcastle disease, bovine viral diarrhea (BVD), equine infectious anemia (EIA), and rinderpest.

According to some of any of the embodiments described herein, the virus is an RNA virus.

According to some of any of the embodiments described herein, the virus is coronavirus, rhabdovirus and/or reovirus.

According to specific embodiments, the virus is a coronavirus.

As used herein, “Coronavirus” refers to enveloped positive-stranded RNA viruses that belong to the family Coronaviridae and the order Nidovirales. Non-limiting examples of coronaviruses include SARS-CoV, SARS-CoV-2, and MERS.

As used herein and in the art, the phrase “viral biomarker” describes a biological molecule found in a biological system, for example, in a blood, other body fluids, and/or tissues of a subject, which is indicative of a presence of a virus and/or a progress of a viral infection in the subject. Viral biomarkers include various types of biomolecules that are specifically associated with the presence of a respective virus in a biological sample and are therefore typically indicative of the existence of a respective viral infection in a subject from which a biological sample is drawn, and may also be indicative of the progress of the viral infection. According to some of any of the embodiments described herein, the viral biomarker is specific to a certain virus or to a certain form of a virus, and is also referred to herein as a viral specific agent or as a viral specific biomarker.

Non-limiting examples of viral biomarkers include viral proteins, viral nucleic acids, metabolites, lipids, and/or other molecules that are associated with the viral pathogen or the host's response to the infection (host response biomarkers).

Herein and in the art, the phrase “viral proteins” describes specific proteins produced by the virus during its replication cycle. For instance, the hepatitis B surface antigen (HBsAg) is a biomarker for hepatitis B infection.

Herein and in the art, the phrase “viral nucleic acids” describes viral DNA or RNA, or fragments thereof, that can be directly detected in the host's cells or body fluids. For example, the presence of HIV RNA in blood is a biomarker for HIV infection.

Herein and in the art, the phrase “host response biomarkers” describes biomarkers that are a result of the host's immune response to the viral infection, such as the production of specific cytokines, antibodies or the activation of certain immune cells.

Non-limiting examples of viruses, the viral infection or disease associated therewith, and corresponding viral biomarkers which their presence and/or (e.g., elevated) level is indicative of the viral infection or the associated disease are set forth in Table B below.

TABLE B

| Viral specie | Disease | Exemplary viral biomarker |
|---|---|---|
| Human immunodeficiency | HIV infection, acquired | Reverse transcriptase; p24 antigen |
| virus (HIV) | immunodeficiency | |
| | syndrome (AIDS) | |
| Hepatitis B virus (HBV) | Hepatitis B | DNA polymerase |
| Hepatitis C virus (HCV) | Hepatitis C | NS3/4A protease |
| Influenza virus | Influenza (flu) | Neuraminidase |
| Human papillomavirus | Cervical cancer, other | E6 and E7 proteins (interact with |
| (HPV) | HPV-related cancers | enzymes like p53 and |
| | | retinoblastoma protein) |
| Herpes simplex virus | Herpes simplex | Thymidine kinase |
| (HSV) | | |
| Cytomegalovirus (CMV) | Cytomegalovirus infection | UL97 kinase |
| SARS-COV-2 | COVID-19 | Main protease (CL^{pro}, SEQ ID NO: |
| | | 1) and papain-like protease (PL^{Pro}) |
| Zaire ebolavirus (Ebola | Ebola virus disease (EVD) | Envelope glycoprotein (GP); VP30 |
| virus; EBOV) | | and VP35 |
| Zika virus | Zika fever | Non-structural protein 5 (NS5) |
| | | polymerase |
| Dengue virus | Dengue fever | NS3 protease |
| Human T-cell leukemia | Adult T-cell leukemia/ | Tax protein |
| virus (HTLV) | lymphoma (ATLL) | |
| West Nile virus (WNV) | West Nile fever, | NS5 polymerase |
| | neuroinvasive disease | |
| Measles virus | Measles | Hemagglutinin (H) protein |
| Rabies virus | Rabies | Glycoprotein (G) |
| Norovirus | Gastroenteritis | RNA-dependent RNA polymerase |
| | | (RdRp) |
| Epstein-Barr virus (EBV) | Infectious mononucleosis, | EBNA-1 (Epstein-Barr nuclear |
| | nasopharyngeal carcinoma, | antigen 1) |
| | Burkitt's lymphoma | |
| Respiratory syncytial virus | Bronchiolitis, pneumonia | F protein (fusion protein) |
| (RSV) | | |
| Rotavirus | Gastroenteritis | VP6 protein |
| Varicella-Zoster virus | Chickenpox, shingles | gE glycoprotein (glycoprotein E) |
| (VZV) | | |
| Coxsackievirus | Hand, foot, and mouth | VP1 protein |
| | disease, myocarditis | |
| Lassa virus | Lassa fever | Glycoprotein complex (GPC) |
| Chikungunya virus | Chikungunya fever | El glycoprotein |
| (CHIKV) | | |
| Hantavirus | Hantavirus pulmonary | Nucleocapsid protein (N protein) |
| | syndrome (HPS), | |
| | hemorrhagic fever with | |
| | renal syndrome (HFRS) | |

The method of the present embodiments comprises a selective interaction between the viral biomarker and an agent that selectively reacts with the viral biomarker, whereby this selective interaction is detectable colorimetrically.

Herein throughout, the phrases “an agent that selectively reacts with the vial biomarker” and an “agent that specifically reacts with the viral biomarker” are used interchangeably, and this agent is also referred to herein, for simplicity, as “selective agent” or simply as “agent”.

As used herein, the phrase “selectively reacts with” refers to a selective and precise interaction between the agent and the viral biomarker, in which the agent binds to the viral biomarker at a much higher level than to another, even structurally or functionally similar, species (e.g., similar biomarkers originating from other viral species, different agents having physically/chemically similar structure). The selectivity can be due to molecular recognition mechanisms, such as those known for biological systems.

In some of any of the embodiments described herein, the agent that selectively reacts with the viral biomarker and the viral biomarker form an affinity pair.

As used herein and in the art, the phrase “affinity pair” refers to two molecules or molecular structures that have a specific and high affinity interaction with each other, enabling them to bind or associate in a selective manner. As known in the art, affinity is typically measured by the equilibrium dissociation constant (Kd), which quantifies the strength of the binding interaction between two molecules (e.g., the agent and the viral biomarker, as described herein). Lower Kd values indicate higher affinity, as they represent stronger and more stable interactions. In the context of the present embodiments, one member of an affinity pair is the viral biomarker and the other one is the agent that selectively reacts with the viral biomarker.

Non-limiting examples of affinity pairs include an enzyme-substrate pair, a polypeptide-polypeptide pair (e.g., a hormone and receptor, a ligand and receptor, an antibody and an antigen, two chains of a multimeric protein), a polypeptide-small molecule pair (e.g., avidin or streptavidin with biotin, enzyme-substrate), a polynucleotide and its cognate polynucleotide such as two polynucleotides forming a double strand (e.g., DNA-DNA, DNA-RNA, RNA-DNA), a polypeptide-polynucleotide pair (e.g., a complex formed of a polypeptide and a DNA or RNA e.g., aptamer), a polypeptide-metal pair (e.g., a protein chelator and a metal ion), a polypeptide and a carbohydrate (leptin-carbohydrate), and the like.

In some of any of the embodiments described herein, the viral biomarker and the agent that selectively reacts with the viral biomarker form an affinity pair, as defined herein, which is characterized by a dissociation constant, Kd, of no more than 1 mM, or no more than 10⁻⁴ M, or no more than 10⁻⁵ M, or no more than 10⁻⁶ M, or no more than 10⁻⁷ M (100 nM), or no more than 108 M (10 nM), or no more than 10⁻⁹ M (1 nM), or no more than 10⁻¹⁰ M, or no more than 10⁻¹² M, and even lower, e.g., as low as 10⁻¹⁵ M.

In some of any of the embodiments described herein, the viral biomarker and the agent that selectively reacts with the viral biomarker form an affinity pair, as defined herein, which is characterized by a dissociation constant (e.g., Kd1), which is lower than a dissociation constant (e.g., Kd2) of an affinity pair formed by the agent that selectively reacts with the viral biomarker and an additional viral biomarker which is analogous to the viral biomarker (e.g., the additional viral biomarker is the same biomarker originating from a different virus, e.g., as demonstrated in Example 4 that follows), such that Kd1 is lower than Kd2 by at least 2-fold, or by at least 3-fold, or by at least 4-fold, or by at least 5-fold, or by at least 10-fold, or by at least 20-fold, or by at least 50-fold, or by at least 2 orders of magnitude, or by at least 3 orders of magnitude, or by at least 4 orders of magnitude, or by at least 5 orders of magnitude, or by at least 6 orders of magnitude, or by at least 9 orders of magnitude, and by even more, e.g., 50 orders of magnitude.

According to some of any of the embodiments described herein, the selective agent is contacted with the sample and thereby interacts with the viral biomarker (if present in the sample).

According to some of any of the embodiments described herein, this interaction generates a (e.g., chemical) species (e.g., a species generated by the reaction between the viral biomarker and the agent that selectively reacts with the viral biomarker as described herein).

According to some of any of the embodiments described herein, the selective agent and the viral biomarker are selected such that a reaction that generates or consumes a (e.g., chemical) species occurs upon the interaction therebetween, and the species or a change in its amount is detectable colorimetrically in the presence of a colorimetric indicator, that is, the consumption or generation of species results in a colorimetric change in the colorimetric indicator, as is described in further detail hereinbelow.

According to some of any of the embodiments described herein, contacting the selective agent and the viral biomarker is such that the selective agent reacts with the viral biomarker so as to generate or consume a (e.g., chemical) species, as described herein.

According to some of any of the embodiments described herein, the selective agent and the viral biomarker are selected such that the selective agent reacts with the viral biomarker so as to generate or consume a (e.g., chemical) species, as described herein.

In some of any of the embodiments described herein, the viral biomarker and the agent that selectively reacts with the viral biomarker are selected capable of generating a (e.g., chemical) species (i.e., a species generated by the reaction between the viral biomarker and the agent that selectively reacts with the viral biomarker) upon a reaction therebetween (between the viral biomarker and the agent that selectively reacts with the viral biomarker). The reaction occurs upon contacting the selective agent with the viral biomarker (e.g., upon contacting a sample as described herein with the selective agent). The reaction can occur at ambient conditions (e.g., ambient temperature and pressure), and/or at physiological temperature. Otherwise, the method further comprises, subsequent or during the contacting, applying conditions that promote the reaction (e.g., temperature, light, etc.).

In some of any of the embodiments described herein, the reaction between the viral biomarker and the agent that selectively reacts with the viral biomarker generates (i.e., produces) a (e.g., chemical) species (i.e., a species generated by the reaction between the viral biomarker and the agent that selectively reacts with the viral biomarker). The species can be a product, a side-product or a by-product of the reaction.

Non-limiting examples of reactions between the viral biomarker and the agent that selectively reacts with the viral biomarker include an acid-base reaction, a redox reaction, a complexation reaction, ligand-receptor binding, and a chemiluminescent reaction.

In some of any of the embodiments described herein, the species generated or consumed by the reaction between the viral biomarker and the agent that selectively reacts with the viral biomarker is a chemical species, for example, a proton or a photon or an electron. Non-limiting examples of species generated by the reaction between the viral biomarker and the agent that selectively reacts with the viral biomarker include, for example, acidic species (e.g., protons or other species having a pKa value of up to 7.0, or up to 6.5), basic species (e.g., hydroxide (OH⁻) or any other species having a pKa value of at least 7.0, or at least 7.5), an organic complex, an inorganic complex, O₂, electrons, and photons.

In some of any of the embodiments described herein, a reaction of the viral biomarker and the agent that selectively reacts with the viral biomarker is performed in the presence of a colorimetric indicator and results in a colorimetric change in the colorimetric indicator.

As used herein, the phrase “colorimetric change” encompasses any change which is visual as perceived by the human visual system (e.g., has a maximal wavelength which is within the visual spectrum) and/or is detectable using an optical device (e.g., a fluorescent device, a spectrophotometer, a colorimeter).

In some of any of the embodiments described herein, the viral biomarker and the agent that selectively reacts with the viral biomarker as described herein in any of the respective embodiments are selected such that the colorimetric change as described herein is a result of a presence of the species generated by the reaction therebetween (i.e., between the viral biomarker and the agent that selectively reacts with the viral biomarker).

In some of any of the embodiments described herein, the species generated by the reaction between the viral biomarker and the agent that selectively reacts with the viral biomarker is characterized by a visual color or a visual change in color (i.e., as perceived by the human visual system). In such embodiments, the colorimetric indicator can be simply the medium or matrix in or into which the contacting is effected.

In some of any of the embodiments described herein, the species generated by the reaction between the viral biomarker and the agent that selectively reacts with the viral biomarker is characterized by a color or a change in color (colorimetric change) which cannot be perceived visually by a human being or is not characterized by a color or a change in color (colorimetric change). In some such embodiments, the reaction between the viral biomarker and the agent that selectively reacts with the viral biomarker further comprises a colorimetric indicator, which exhibits a colorimetric change in response to the reaction between the viral biomarker and the agent that selectively reacts with the viral biomarker, for example, in response to the generation or consumption of the species as described herein, as is described in further detail hereinunder.

According to some of any of the embodiments described herein, the selective agent and the viral biomarker form an affinity pair as described herein in any of the respective embodiments, and in some of these embodiments, the affinity pair is such that when formed, a species as described herein is generated or consumed. The method therefore is colorimetrically responsive to the generation or consumption of the species.

In some embodiments, the affinity pair comprises an enzyme and its substrate, which generate or consume a species during the enzymatic catalytic reaction.

According to some of any of the embodiments described herein, the viral biomarker is an enzyme, for example, an enzyme specific to a certain virus. According to some of these embodiments, the selective agent is a substrate specific to the enzyme. In view of the catalytic nature of the enzymatic reaction, the reaction requires only catalytic amount of the enzyme and hence occurs also in the presence of a very low concentration of the viral biomarker, and thereby provides a sensitive detection thereof.

In some embodiments, the viral biomarker is an enzyme and the agent that selectively reacts with the viral biomarker is a substrate having a high affinity, as defined herein, to the enzyme. It is to be noted that embodiments where the viral biomarkers is a substrate specific to an enzyme and the selective agent is that enzyme are also contemplated. In such embodiments, one can select as the selective agent an enzyme suitable to the viral biomarker.

In some of any of the embodiments described herein, the viral biomarker is a viral enzyme and the agent that selectively reacts with the viral biomarker is a substrate specific to the viral enzyme. In some embodiments, the viral biomarker is a viral enzyme and the agent that selectively reacts with the viral biomarker is a substrate having a high affinity, as defined herein, to the viral enzyme.

According to some of any of the embodiments described herein, the viral biomarker is indicative of an active viral invention, that is, it is a biological species that participates in the life-cycle of the virus (e.g., in viral replication).

According to some of any of the embodiments described herein, the viral biomarker is a non-structural protein.

According to some of any of the embodiments described herein, the viral biomarker is an enzyme that is indicative of an active viral invention, that is, it participates in the life cycle of the virus (e.g., in viral replication).

According to some of any of the embodiments described herein, the viral biomarker is an enzyme that is indicative of an active viral infection, that is, it participates in the life cycle of the virus (e.g., in viral replication) and is a non-structural enzyme.

When the viral biomarker is an enzyme, in some embodiments, the enzyme catalyzes a reaction that generates or consumes a species as described herein, for example, protons and/or electrons. Such enzymes can be, for example, redox enzymes, that generate or consume electrons, or proteolytic enzymes, that can generate or consume protons.

According to some of any of the embodiments described herein, the viral biomarker is a proteolytic enzyme.

As used herein and in the art, the phrase “proteolytic enzyme” (also being referred to interchangeably as a protease) is a type of enzyme that catalyzes the breakdown of proteins into smaller peptides or amino acids. Proteolytic enzymes achieve this by cleaving the peptide bonds between amino acids in the protein chain. Non-limiting examples include endopeptidases, exopeptidases, serine proteases, cysteine proteases, aspartic proteases, and metalloproteases.

In some of any of the embodiments described herein, the viral biomarker is a proteolytic enzyme and the agent that selectively reacts with the viral biomarker is a substrate specific to the viral enzyme. In some embodiments, the viral biomarker is a proteolytic enzyme and the agent that selectively reacts with the viral biomarker is a substrate having a high affinity, as defined herein, to the proteolytic enzyme.

In some of any of the embodiments described herein, the viral biomarker is a viral proteolytic enzyme and the agent that selectively reacts with the viral biomarker is a substrate specific to the viral proteolytic enzyme. In some embodiments, the viral biomarker is a viral proteolytic enzyme and the agent that selectively reacts with the viral biomarker is a substrate having a high affinity, as defined herein, to the viral proteolytic enzyme.

In some of any of the embodiments described herein, the viral biomarker is a viral proteolytic enzyme specific to a certain virus.

In some of any of the embodiments described herein, the viral proteolytic enzyme and its substrate are selected such that a change in pH of a medium in which the enzymatic reaction (cleavage) occurs. A pH change (as a result of generation or consumption of protons) can be a result of formation of acidic cleavage products, for example, protons generated by the reaction or amino acid residues featuring a carboxylic acid formed by the cleavage.

According to some of any of the embodiments described herein, the viral biomarker is a viral proteolytic enzyme, as described herein, that is indicative of an active viral invention, that is, it participates in the life-cycle of the virus (e.g., in viral replication), and the agent that selectively reacts with the viral biomarker is a substrate specific to the viral proteolytic enzyme.

According to some of any of the embodiments described herein, the viral biomarker is a viral proteolytic enzyme, as described herein, that is indicative of an active viral invention, that is, it participates in the life-cycle of the virus (e.g., in viral replication) and is a non-structural enzyme, and the agent that selectively reacts with the viral biomarker is a substrate specific to the viral proteolytic enzyme. According to some of any of the embodiments described herein, the viral biomarker is a viral proteolytic enzyme, as described herein in any of the respective embodiments, which is present in a saliva of subject having a viral infection.

According to some of any of the embodiments described herein, the viral biomarker is a proteolytic enzyme specific to SARS-CoV-2, that is, a SARS-CoV-2-specific proteolytic enzyme. According to some of any of the embodiments described herein, the viral biomarker is a proteolytic enzyme specific to SARS-CoV-2 which is present in the saliva of the subject. Non-limiting examples of SARS-CoV-2-specific proteolytic enzymes include 3CL^{pro} (SEQ ID NO: 1) and PL^{pro}.

As used herein and in the art, the term “3CL^{pro}” refers to 3 chymotrypsin-like proteinase, PDB: 6LZE, having an EC no. 3.4.22.69. It is a cysteine protease which is the main protease found in coronaviruses. It cleaves the coronavirus polyprotein at eleven conserved sites. It has a cysteine-histidine catalytic dyad at its active site and cleaves a Gln-(Ser/Ala/Gly) peptide bond.

In some of any of the embodiments described herein, the viral biomarker is a SARS-CoV-2 proteolytic enzyme and the agent that selectively reacts with the viral biomarker is a substrate specific to the SARS-CoV-2 proteolytic enzyme. In some embodiments, the viral biomarker is a SARS-CoV-2 proteolytic enzyme and the agent that selectively reacts with the viral biomarker is a substrate having a high affinity, as defined herein, to the SARS-CoV-2 proteolytic enzyme.

In some of any of the embodiments described herein, the viral biomarker is an enzyme and the agent that selectively reacts with the viral biomarker is a peptide substrate specific to the enzyme.

The peptide substrate can be of from 3 to 100, or from 3 to 80, or from 3 to 60, or from 3 to 50, or from 3 to 40, or from 3 to 30, or from 3 to 20, or from 4 to 100, or from 4 to 80, or from 4 to 60, or from 4 to 50, or from 4 to 40, or from 4 to 30, or from 4 to 20, or from 5 to 100, or from to 80, or from 5 to 60, or from 5 to 50, or from 5 to 40, or from 5 to 30, or from 5 to 20, or from to 100, or from 10 to 80, or from 10 to 60, or from 10 to 50, or from 10 to 40, or from 10 to 30, or from 10 to 20, amino acids. The peptide substrate is a peptide as described herein.

In some of any of the embodiments described herein, the viral biomarker is a viral enzyme and the agent that selectively reacts with the viral biomarker is a peptide substrate specific to the viral enzyme. In some embodiments, the viral biomarker is a viral enzyme and the agent that selectively reacts with the viral biomarker is a peptide substrate having a high affinity, as defined herein, to the viral enzyme.

In some of any of the embodiments described herein, the viral biomarker is a viral proteolytic enzyme and the agent that selectively reacts with the viral biomarker is a peptide substrate specific to the viral enzyme. In some embodiments, the viral biomarker is a proteolytic enzyme and the agent that selectively reacts with the viral biomarker is a peptide substrate having a high affinity, as defined herein, to the proteolytic enzyme.

In some of any of the embodiments described herein, the viral biomarker is a viral proteolytic enzyme and the agent that selectively reacts with the viral biomarker is a peptide substrate specific to the viral proteolytic enzyme. In some embodiments, the viral biomarker is a viral proteolytic enzyme and the agent that selectively reacts with the viral biomarker is a peptide substrate having a high affinity, as defined herein, to the viral proteolytic enzyme.

In some of any of the embodiments described herein, the viral biomarker is a SARS-CoV-2 proteolytic enzyme and the agent that selectively reacts with the viral biomarker is a peptide substrate specific to the SARS-CoV-2 proteolytic enzyme. In some embodiments, the viral biomarker is a SARS-CoV-2 proteolytic enzyme and the agent that selectively reacts with the viral biomarker is a peptide substrate having a high affinity, as defined herein, to the SARS-CoV-2 proteolytic enzyme.

According to some of any of the embodiments described herein, the agent that selectively reacts with the viral biomarker is a peptide substrate that the SARS-CoV-2-specific proteolytic enzyme, as described herein in any of the respective embodiments, selectively cleaves.

According to some of any of the embodiments described herein, the agent that selectively reacts with the viral biomarker is a peptide.

According to some of any of the embodiments described herein, the agent that selectively reacts with the viral biomarker is a peptide substrate that 3CL^{pro} selectively cleaves. According to some of any of the embodiments described herein, the agent that selectively reacts with the viral biomarker is a peptide substrate that SARS-CoV-2 3CL^{pro} selectively cleaves.

According to some of any of the embodiments described herein, the peptide substrate as described herein in any of the respective embodiments is cleavable by a viral proteolytic enzyme as described herein in any of the respective embodiments.

According to some of any of the embodiments described herein, the peptide substrate as described herein in any of the respective embodiments is cleavable by 3CL^{pro}.

According to some of any of the embodiments described herein, the peptide substrate as described herein in any of the respective embodiments is cleavable by the SARS-CoV-2-specific proteolytic enzyme 3CL^{pro}. Non-limiting examples include a peptide having any one of SEQ ID NOs: 2 and 10-30.

According to some of any of the embodiments described herein, the peptide substrate is selected such that its cleavage by the proteolytic enzyme results in a colorimetric change in the colorimetric indicator as described herein. For example, the peptide substrate is selected such that its cleavage by the proteolytic enzyme results in a generation or consumption of a species as described herein.

As used herein, the phrase “colorimetric indicator” refers to a compound capable of changing its color to provide a colorimetric change, for example, from colored to colorless, from colorless to colored, or from one color (e.g., blue) to another color (e.g., yellow). Non-limiting examples include pH indicators, redox indicators (e.g., methylene blue, diphenylamine, ferroin), complexometric indicators (e.g., eriochrome black T, calmagite, murexide), enzyme indicators (e.g., X-gal, for β-galactosidase activity; tetramethylbenzidine (TMB), for peroxidase activity), moisture indicators (e.g., cobalt chloride paper), ionic/specific indicators (e.g., diphenylcarbazone, for mercury; potassium ferricyanide, for iron), and dual indicators (e.g., pH- and redox-indicators such as indigo carmine).

According to the present embodiments, the colorimetric indicator is capable of changing its color to provide a colorimetric change in response to the interaction between the viral biomarker and the selective agent, for example, in response to the generation or consumption of a species as described herein.

In some of any of the embodiments described herein, the colorimetric change in the colorimetric indicator is in response to an interaction of the colorimetric indicator with a (e.g., chemical) species (i.e., a species generated or consumed by the reaction between the viral biomarker and the agent that selectively reacts with the viral biomarker as described herein).

According to some of any of the embodiments described herein, the colorimetric indicator is selected such that a cleavage of the peptide substrate by the proteolytic enzyme results in a colorimetric change in the colorimetric indicator as described herein. For example, the colorimetric indicator is selected such that generation or consumption of the species results in a change in its color (the colorimetric change).

In some of any of the embodiments described herein, the species generated or consumed by the reaction between the viral biomarker and the agent that selectively reacts with the viral biomarker are electrons. According to some of any of the embodiments described herein, the reaction of the agent with the viral biomarker is a redox reaction, and the colorimetric indicator is a redox indicator.

In some of any of the embodiment, the species generated by the reaction between the viral biomarker and the agent that selectively reacts with the viral biomarker are photons. The colorimetric indicator can be a luminescent indicator. In any of the embodiments described herein, a colorimetric indicator is selected in accordance with the species that is formed or consumed when the viral biomarker and the selective agent contact one another.

In some of any of the embodiments described herein, the species generated or consumed by the reaction between the viral biomarker and the agent that selectively reacts with the viral biomarker are protons.

According to some of any of the embodiments described herein, the reaction of the agent with the viral biomarker results in a pH change of an aqueous solution which is in contact with the viral biomarker and/or the selective agent, and the colorimetric indicator is a pH indicator as described herein.

As used herein and in the art, the phrase “pH indicator” (also known as an “acid base indicator”) describes a substance that changes its color in response to a change in the concentration of protons in an aqueous solution, that is a change in the acidity or alkalinity of a solution (the pH of a solution), thereby providing a visual means to determine the pH level of the solution. Non-limiting examples of pH indicators include phenolphthalein (pKa of about 9.4), thymol blue (two pKas of about 1.7 and 8.9), cresol red (two pKas of about 1.0 and 8.3), bromothymol blue (pKa of about 7.1), methyl red (pKa of about 5.0), bromocresol green (pKa of about 4.7), methyl orange (pKa of about 3.4), and litmus (comprising lichens-based dyes; pKa of about 6.5).

In some of any of the embodiments described herein, the colorimetric indicator exhibits more than one colorimetric change (i.e., more than two changes in color) in response to the reaction between the viral biomarker and the agent that selectively reacts with the viral biomarker. In some embodiments, the colorimetric indicator is an acid-base indicator which is characterized by more than one pKa value. In some embodiments, the colorimetric indicator has dual activity (e.g., is both an acid-base and a redox indicator). In some embodiments, the colorimetric indicator comprises more than one indicator (e.g., two pH indicators, or a combination of a pH indicator and a redox indicator).

According to exemplary embodiments, the selective agent is a peptide substrate of a viral proteolytic enzyme which is indicative of a viral infection (e.g., of an active viral active infection as described herein), which is selected such that its cleavage by the enzyme results in a change of the pH of the medium where the cleavage occurs, as described and explained herein in any of the respective embodiments and any combination thereof. According to some of these embodiments, the colorimetric agent is a pH indicator.

According to exemplary embodiments, the peptide substrate is of a SARS-CoV-2-specific proteolytic enzyme, as described herein in any of the respective embodiments, and the colorimetric indicator is a pH indicator.

According to some of any of the embodiments described herein, the contacting is effected in a solution, preferably an aqueous solution, for example, a buffered aqueous solution.

According to some of any of the embodiments described herein, the contacting is effected by contacting a (e.g., aqueous, e.g., buffered aqueous) solution that comprises the agent that selectively reacts with the viral biomarker as described herein in any of the respective embodiments, and the colorimetric indicator as described herein in any of the respective embodiments; with the (e.g., biological) sample as described herein in any of the respective embodiments. For example, a saliva sample is drawn from the subject by means of, e.g., a swab, and the swab is contacted with the aqueous solution.

According to some of any of the embodiments described herein, the contacting is effected by contacting the sample with a (e.g., aqueous or buffered aqueous) solution that comprises the agent that selectively reacts with the viral biomarker as described herein in any of the respective embodiments, and adding to the solution the colorimetric indicator as described herein in any of the respective embodiments. For example, a saliva sample is drawn from the subject by means of e.g., a swab, and the swab is contacted with the aqueous solution comprising the selective agent. The colorimetric indicator, optionally as an (e.g., buffered) aqueous solution or as a solid matrix as described herein, is then added to the solution.

According to some of any of the embodiments described herein, the contacting is effected by contacting the sample with a (e.g., aqueous or buffered aqueous) solution that comprises the colorimetric indicator, as described herein in any of the respective embodiments, and the agent that selectively reacts with the viral biomarker as described herein in any of the respective embodiments is then added to the solution. For example, a saliva sample is drawn from the subject by means of e.g., a swab, and the swab is contacted with the aqueous solution comprising the colorimetric indicator. The selective agent is then added to the solution.

According to some of any of the embodiments described herein, the contacting is effected by contacting the sample with a (e.g., aqueous or buffered aqueous) solution that comprises both the agent that selectively reacts with the viral biomarker as described herein in any of the respective embodiments, and the colorimetric indicator as described herein in any of the respective embodiments. The colorimetric indicator can be in a form of a solution or as a solid matrix, as described herein. For example, a saliva sample is drawn from the subject by means of e.g., a swab, and the swab is contacted with the aqueous solution comprising the selective agent and the colorimetric indicator.

According to any of these embodiments, a change in the color of the colorimetric indicator, a colorimetric change, is indicative of a presence of the viral biomarker in the sample, and/or of a presence of a (e.g., active) viral infection in a subject from which a biological sample is drawn.

According to some of any of the embodiments described herein, the contacting is effected at room temperature or ambient temperature, for example, of from about 15 to about 30, or from about 15 to about 25, or from about 20 to about 30, or from about 20 to about 25, ° C. Alternatively, the contacting is effected at physiological temperature (of about 37° C.).

According to some of any of the embodiments described herein, the colorimetric indicator is or comprises a solid matrix.

According to some of any of the embodiments described herein, the solid matrix is selected capable of absorbing therein and/or thereon (covalently or non-covalently binding to the surface of the solid matrix or embedded within the solid matrix) the viral biomarker as described herein in any of the respective embodiments, while maintaining its biological activity. In exemplary embodiments, the solid matrix is inert to the viral biomarker, such that it is incapable of reacting with the viral biomarker and does not affect its structure and/or activity.

According to some of any of the embodiments described herein, the solid matrix is selected capable of absorbing therein and/or thereon (as described herein) the sample as described herein in any of the respective embodiments.

Herein, the phrase “absorb the sample to the solid matrix” refers to the process by which the sample, or at least the viral biomarker (if such is present in the sample) is taken up and retained within the internal structure of the solid matrix (absorbed in) and/or adheres to the surface of the solid matrix (absorbed on). This phrase encompasses both internal incorporation of the sample by the solid matrix and surface attachment of the sample to the solid matrix material, and encompasses a dried solid matrix (i.e., dried following the absorption of, e.g., the agent thereto) or a wet solid matrix (i.e., undried following the absorption of, e.g., the agent thereto). According to some of any of the embodiments described herein, the solid matrix is porous, or at least features a porous surface, which allows absorbing thereto the sample. According to some of any of the embodiments described herein, the solid matrix is fibrous, or at least features a fibrous surface, which allows absorbing thereto the sample. In some embodiments, porous and/or fibrous structures feature pores or void having an average size (e.g., diameter) in the micrometer and/or nanometer scale, so as to allow efficient entrapment or entanglement of the viral biomarker therewith.

According to exemplary embodiments, the method is effected by contacting the solid matrix with the sample, so as to provide a solid matrix having therein and/or thereon (as described herein) the sample or at least the viral biomarker.

According to some of any of these embodiments, the solid matrix having the sample or at least the viral biomarker absorbed thereto, is contacted with the selective agent. In some of these embodiments, the solid matrix is contacted with the selective agent. According to some of these embodiments, the solid matrix is contacted with an aqueous solution that comprises the selective agent. According to some of these embodiments, the solid matrix exhibits a colorimetric change if the sample comprises the viral biomarker.

Contacting the solid matrix with the sample and with the selective agent can be performed sequentially, in any order, or concomitantly.

According to exemplary embodiments, the method is effected by contacting the solid matrix with the sample, so as to provide a solid matrix having absorbed therein and/or thereon (as described herein) the sample or at least the viral biomarker, subsequently contacting the solid matrix with an aqueous solution e.g., a buffered aqueous solution), and then contacting the solid matrix with the selective agent (e.g., with an aqueous solution (e.g., a buffered aqueous solution) that comprises the selective agent).

According to exemplary embodiments, the method is effected by contacting the solid matrix with the selective agent, for example, with an aqueous solution that comprises the selective agent, so as to provide a solid matrix having the selective agent absorbed thereto. In cases when an aqueous solution of the active agent is used, the solid matrix can optionally be dried before the method proceeds. According to these embodiments, the method proceeds by contacting the solid matrix having the selective agent absorbed thereto with the sample, so as to have a solid matrix having the selective agent and the sample absorbed thereto. According to some of these embodiments, the solid matrix exhibits a colorimetric change if the sample comprises the viral biomarker. Alternatively, an aqueous solution (e.g., a buffered aqueous solution) is contacted with the solid matrix to thereby exhibit the colorimetric change, either before, during or subsequent to contacting the solid matrix with the sample.

According to exemplary embodiments, the method is effected by contacting the solid matrix with the selective agent, for example, with an aqueous solution that comprises the selective agent, so as to provide a solid matrix having the selective agent absorbed thereto. In cases when an aqueous solution of the active agent is used, the solid matrix can optionally be dried before the method proceeds. According to these embodiments, the method proceeds by contacting the solid matrix having the selective agent absorbed thereto with the sample, so as to have a solid matrix having the selective agent and the sample absorbed thereto. According to some of these embodiments, the method proceeds by contacting the solid matrix having the selective agent and the sample absorbed thereto with an aqueous solution (e.g., a buffered aqueous solution as described herein). According to these embodiments, the solid matrix exhibits a colorimetric change if the sample comprises the viral biomarker.

According to some of any of the embodiments described herein, when the solid matrix is contacted with the sample, the sample can be used per se, e.g., a biological sample drawn from the subject as described herein, preferably without being further processed or treated, or can be, for example, an aqueous solution (e.g., a buffered aqueous solution) comprising the sample.

According to some of any of the embodiments described herein, an aqueous solution (e.g., a buffered aqueous solution), for example, an aqueous solution (e.g., a buffered aqueous solution) that is contacted with the solid matrix, with or without the selective agent and/or the sample, participates in the colorimetric change.

According to some of any of the embodiments described herein, the colorimetric indicator is a pH indicator (e.g., pH paper as described herein), and the reaction between the selective agent and the viral biomarker generates or consumes protons or results in a pH change in the medium (e.g., aqueous solution) where it occurs. According to some of these embodiments, an initial pH of the aqueous solution (e.g., a buffered aqueous solution) is different from the pH of the aqueous solution after the selective agent reacts with the viral biomarker, such that the pH of the aqueous solution changes in response to the reaction between the viral biomarker and the selective agent, and this change represents the colorimetric change.

According to some of these embodiments, the pH is of the (e.g., aqueous) solution that comprises the agent that selectively reacts with the viral biomarker decreases upon the reaction between the agent and a pre-determined threshold amount of the viral biomarker in the sample.

According to some of any of the embodiments described herein, the aqueous solution (e.g., a buffered aqueous solution) features an initial (before contacting the components) pH that differs from a pH of an aqueous solution after the selective agent and the viral biomarker interact (an interaction the result in decrease of the pH) by at least 0.3 pH unit, or by at least 0.4 pH unit, or by at least 0.5 pH unit, or by at least 0.6 pH unit, or by at least 0.7 pH unit, or by at least 0.8 pH unit, or by at least 0.9 pH unit, or by at least one pH unit, from example, by 0.3-4, or by 0.3-3, or 0.3-2, or 0.3-1, or 0.3-0.5, or 0.4-3, or 0.4-2, or 0.4-2, or 0.5-3, or 0.5-2, or 0.5-1, or 1-3, or 1-2, pH units, including any intermediate values and subranges therebetween.

According to some of any of the embodiments described herein, a pH of the (e.g., aqueous; or buffered aqueous solution) solution that comprises the agent that selectively reacts with the viral biomarker decreases upon the reaction between the viral biomarker and the agent that selectively reacts with the viral biomarker.

According to some of these embodiments, the colorimetric indicator is a pH indicator, and the aqueous solution is characterized by an (initial) pH in the range of from 7.5 to 14, or from 7.5 to 13, or from 7.5 to 12, or from 7.5 to 11, or from 7.5 to 10, or from 7.5 to 9, or from 7.5 to 8.5, or from 8 to 14, or from 8 to 13, or from 8 to 12, or from 8 to 11, or from 8 to 10, or from 8 to 9, or is 8.5, including any intermediate values and subranges therebetween.

According to some of any of the embodiments described herein, the aqueous solution has a pH higher by at least 0.3 pH unit, or by at least 0.4 pH unit, or by at least 0.5 pH unit, or by at least 0.6 pH unit, or by at least 0.7 pH unit, or by at least 0.8 pH unit, or by at least 0.9 pH unit, or by at least one pH unit, for example, 0.3-4, or by 0.3-3, or 0.3-2, or 0.3-1, or 0.3-0.5, or 0.4-3, or 0.4-2, or 0.4-2, or 0.5-3, or 0.5-2, or 0.5-1, or 1-3, or 1-2, pH units, including any intermediate values and subranges therebetween, from a pH of the (e.g., aqueous) solution that comprises the agent that selectively reacts with the viral biomarker decreases upon the reaction between the agent and a pre-determined threshold amount of the viral biomarker in the sample.

According to some of any of the embodiments described herein, the pre-determined threshold amount of the viral biomarker in the sample (i.e., a lowest concentration of the viral biomarker detectable by the system or test strip as described herein in any of the respective embodiments) is about 0.01 μg ml⁻¹, or is about 0.02 μg ml⁻¹, or is about 0.03 μg ml⁻¹, or is about 0.04 μg ml⁻¹, or is about 0.05 μg ml⁻¹, or is about 0.06 μg ml⁻¹, or is about 0.07 μg ml⁻¹, or is about 0.08 μg ml⁻¹, or is about 0.09 μg ml⁻¹, or is about 0.1 μg ml⁻¹, or is about 0.11 μg ml⁻¹, or is about 0.15 μg ml⁻¹, or is about 0.2 μg ml⁻¹, or is about 0.5 μg ml⁻¹, or is about 1 μg ml⁻¹, or is about 10 μg ml⁻¹, including any intermediate values and subranges therebetween.

According to some of any of the embodiments described herein, the method comprises contacting the (e.g., biological) sample as described herein in any of the respective embodiments with the solid matrix, to thereby absorb the sample to (i.e., in and/or on) the solid matrix; and contacting the solid matrix (e.g., a pH paper) having the sample absorbed thereto with the aqueous solution that comprises the agent that selectively reacts the viral biomarker as described herein in any of the respective embodiments, wherein a colorimetric change (e.g., the colorimetric change as described herein in any of the respective embodiments) in the solid matrix (e.g., pH paper) is indicative of a presence and/or level of the viral biomarker in the sample.

In some of any of the embodiments described herein, the solid matrix having the sample absorbed thereto is dried (e.g., air-dried) prior to its contact with an aqueous solution (e.g., an aqueous solution that comprises the selective agent, and/or an aqueous solution that features pH as described herein).

According to some of any of the embodiments described herein, the solid matrix has absorbed therein and/or thereon (as described herein) the agent that selectively reacts with the viral biomarker as described herein in any of the respective embodiments.

According to some of any of the embodiments described herein, the solid matrix has absorbed therein and/or thereon (as described herein) the peptide substrate as described herein in any of the respective embodiments.

In some of any of the embodiments described herein, the solid matrix having the selective agent absorbed thereto is dried (e.g., air-dried) prior to its contact with the sample or with an aqueous solution (e.g., an aqueous solution that comprises the sample, and/or an aqueous solution that features pH as described herein). According to some of any of the embodiments described herein, when a solid matrix having the selective agent absorbed thereto (e.g., dried) is used, its contact with a sample as described herein can be performed immediately after its preparation, or subsequent to its preparation, for example, within a time period of at least one hour, at least one day, at least one week, at least one month, and even longer time periods.

According to some of any of the embodiments described herein, the reaction of the viral biomarker and the selective agent results in a change in pH of an aqueous solution, and the solid matrix is or comprises a solid pH indicator, for example, a pH paper.

As used herein and in the art, the phrase “pH paper” refers to a type of solid matrix (e.g., a paper, a litmus paper) that is impregnated with a mixture of pH-sensitive dyes. It is used as an indicator to measure the acidity or alkalinity of a solution. Upon dipping the pH paper in a solution, it may change its color depending on the pH level of the solution, providing a visual representation of its acidity or basicity. The resulting color is typically compared to a standard color chart (i.e., a look-up table) to determine the precise pH value. The color can alternatively analyzed by other means, as described hereinunder.

Any commercially available pH paper can be used. Preferably, a pH paper that is fibrous, porous or otherwise absorptive is selected.

According to some of any of the embodiments described herein, the pH paper is characterized by a detectable pH range of from 1 to 14, or from 1 to 10, or from 1 to 9, or from 5 to 14, or from 5 to 10, or from 5 to 9, or from 6 to 10, or from 6 to 9, or from 6 to 8, or from 6.4 to 8, including any intermediate values and subranges therebetween.

According to some of any of the embodiments described herein, the colorimetric change comprises a change in the color of the pH paper.

According to some of these embodiments, the colorimetric change reflects a change in a color of a pH paper that is contacted with an aqueous solution as described herein in any of the respective embodiments, that is, an aqueous solution that features a pH that differs from the pH that results from the reaction of the viral biomarker and the selective agent.

According to some of these embodiments, the aqueous solution and the pH paper together comprise a colorimetric pH indicator.

According to some of any of the embodiments described herein, determining the level of the colorimetric change is performed immediately after the colorimetric indicator, the sample, and the selective agent, and optionally an aqueous solution if such is used, are all contacted together, or within a time period of from about 0.5 minutes to 5 minutes, or from 0.5 minutes to 3 minutes, or from 1 minute to 3 minutes, or can be performed 1, 2, 3, or more hours, thereafter.

According to some of any of the embodiments described herein, determining the level of the colorimetric change comprises visually correlating the color that is formed upon the colorimetric change with a (e.g., printed, look-up) table of possible visual colors, so as to determine the presence and/or amount of the viral biomarker in the sample and/or the viral infection in the subject as described herein based on the formed color.

According to some of any of the embodiments described herein, determining the level of the colorimetric change comprises converting a color that forms upon the colorimetric change to a RGB signal.

As used herein and in the art, the phrase “RGB signal” describes a representation of color information in terms of the intensities of red, green, and blue light, as perceived by the human visual system or by an optical device, wherein each value ranges from 0 to 255 and signifies the intensity of the respective color component.

Herein, the phrase “converting a color ( . . . ) to a RGB signal” refers to any process for translating a given color (i.e., a color that forms upon the colorimetric change), into its corresponding RGB values. Non-limiting examples for translating a color to RGB include using a color chart, using a colorimeter or a spectrophotometer, and using a digital camera (e.g., a smartphone camera).

When using a digital camera, translating a color to RGB signal further includes extracting RGB values from the photo using an image editing software (e.g., Adobe Photoshop™, Microsoft Paint™) or online tools in combination with a respective color picker tool.

A colorimeter typically provides color measurements in non-RGB color space (e.g., CIELAB or CIE XYZ color space). Converting it to RGB values can be performed using a conversion tool which is typically included in the software provided by the colorimeter manufacturer.

A spectrophotometer provides spectral data, often in the form of reflectance or transmittance values. These can be converted to RGB values, e.g., using the stepwise conversion: (i) to a non-RGB color space ((e.g., CIELAB or CIE XYZ color space), which is typically included in the software provided by the spectrophotometer manufacturer, followed by (ii) its conversion to RGB using software and/or online tools, as described herein.

Alternatively, one can translate a color to RGB signal using a color chart, to visually correlate the color that is formed upon the colorimetric change with a (e.g., printed) table of possible visual colors, and to determine the RGB signal as described herein based on the formed color.

According to some of any of the embodiments described herein, determining the level of the colorimetric change is performed based on a look-up table (e.g., a color chart as described herein) that correlates between an amount of the viral biomarker and the color that is formed upon the colorimetric change.

According to some of any of the embodiments described herein, the RGB signal is a B/G signal.

According to some of any of the embodiments described herein, the RGB signal is processed by a signal processor, which is in communication (e.g., computational communication) with the look-up table. According to some of these embodiments, once the RGB signal correlates with a color that is indicative of a presence of the viral biomarker, a visual or acoustic signal is generated.

According to exemplary embodiments, a method as described herein is for determining a presence and/or a level of a viral biomarker by determining a colorimetric change that results from a change in pH, using a pH indicator as described herein.

The pH indicator can be an aqueous solution. The pH indicator can be a solid matrix that is contacted or has absorbed thereto an aqueous solution that comprises a pH indicator (which changes its color in response to a change in the pH of the solution). The pH indicator can be a pH paper that changes its color in response to a change in pH of an aqueous solution that contacts it. The pH indicator can be a solid matrix having absorbed thereto a pH indicator solution.

According to exemplary embodiments, the pH indicator is or comprises a pH solid indicator, for example, a pH paper.

According to exemplary embodiments, a method as described herein is for determining a presence and/or a level of a viral proteolytic enzyme by determining a colorimetric change that results from a change in pH, using a pH indicator as described herein in any of the respective embodiments and any combination thereof. According to these embodiments, the pH change results from a reaction between the viral proteolytic enzyme and substrate thereof (e.g., a peptide substrate).

According to exemplary embodiments, a method as described herein is for determining a presence and/or a level of a biomarker which is specific to a coronavirus, for example, a biomarker specific to SARS-CoV-2. According to these embodiments, the method is effected by determining a colorimetric change that results from a change in pH, using a pH indicator as described herein in any of the respective embodiments and any combination thereof. According to these embodiments, the pH change results from a reaction between the biomarker and a respective selective (e.g., peptide) agent.

According to exemplary embodiments, a method as described herein is for determining a presence and/or a level of a proteolytic enzyme selective to a coronavirus, for example, a SARS-CoV-2-specific proteolytic enzyme. According to these embodiments, the method is effected by determining a colorimetric change that results from a change in pH, using a pH indicator as described herein in any of the respective embodiments and any combination thereof. According to these embodiments, the pH change results from a reaction between the proteolytic enzyme and a substrate thereof (e.g., a peptide substrate).

According to exemplary embodiments, a method as described herein is for determining a presence and/or a level of a 3CL^{pro} enzyme (e.g., having SEQ ID NO:1). According to these embodiments, the method is effected by determining a colorimetric change that results from a change in pH, using a pH indicator as described herein in any of the respective embodiments and any combination thereof. According to these embodiments, the pH change results from a reaction between the 3CL^{pro} enzyme and a substrate thereof (e.g., a peptide substrate), as described herein in any of the respective embodiments any combination thereof, for example, a peptide substrate having SEQ ID NOs: 2 or 10-30.

According to some of these embodiments, the pH indicator comprises a pH paper, as described herein in any of the respective embodiments and any combination thereof.

According to some of these embodiments, the sample is a biological sample, as described herein in any of the respective embodiments and any combination thereof, and in some embodiments, it is a saliva sample.

According to some of these embodiments, the method is effected by contacting the sample with the pH paper, to thereby absorb the sample to the pH paper; and contacting the pH paper having the sample absorbed thereto with an aqueous solution (e.g., buffered aqueous solution) that comprises the agent that specifically reacts with the viral biomarker (e.g., a peptide substrate specific to 3CL^{pro} enzyme as described herein). According to some of these embodiments, the aqueous solution (e.g., buffered aqueous solution) features an initial pH (before contacting the pH paper) as described herein in any of the respective embodiments. According to these embodiments, a colorimetric change in the pH paper is indicative of a presence of the viral biomarker (e.g., 3CL^{pro} enzyme as described herein) in the sample. According to these embodiments, a colorimetric change in the pH paper is indicative of a presence of an active SARS-CoV-2 infection is a subject from the sample is drawn.

According to some of these embodiments, the method is effected by contacting the pH paper with the agent that specifically reacts with the viral biomarker (e.g., a peptide substrate specific to 3CL^{pro} enzyme as described herein), to thereby absorb the agent to the pH paper; and contacting the pH paper having the agent absorbed thereto with the sample. According to some of these embodiments, subsequent to contacting the sample, the pH paper is contacted with an aqueous solution (e.g., buffered aqueous solution) that differs in pH, as described herein in any of the respective embodiments. According to these embodiments, a colorimetric change in the pH paper is indicative of a presence of the viral biomarker (e.g., 3CL^{pro} enzyme as described herein) in the sample. According to these embodiments, a colorimetric change in the pH paper is indicative of a presence of an active SARS-CoV-2 infection is a subject from the sample is drawn.

According to some of these embodiments, the method is effected by contacting the pH paper with an aqueous solution (e.g., buffered aqueous solution) that comprises agent that specifically reacts with the viral biomarker (e.g., a peptide substrate specific to 3CL^{pro} enzyme as described herein), to thereby absorb the agent to the pH paper; and contacting the pH paper having the agent absorbed thereto with the sample. According to some of these embodiments, the aqueous solution (e.g., buffered aqueous solution) features initial pH that differs from the pH upon contacting the selective agent with the viral biomarker, as described herein in any of the respective embodiments. According to these embodiments, a colorimetric change in the pH paper is indicative of a presence of the viral biomarker (e.g., 3CL^{pro} enzyme as described herein) in the sample. According to these embodiments, a colorimetric change in the pH paper is indicative of a presence of an active SARS-CoV-2 infection is a subject from the sample is drawn.

According to some of these embodiments, the method is effected by contacting the pH paper with an aqueous solution (e.g., buffered aqueous solution) that features initial pH that differs from the pH upon contacting the selective agent with the viral biomarker, as described herein in any of the respective embodiments, contacting the pH paper having the aqueous solution absorbed thereto with the sample, and contacting the pH paper having the sample absorbed thereto with the agent that specifically reacts with the viral biomarker (e.g., a peptide substrate specific to 3CL^{pro} enzyme as described herein) or an aqueous solution (e.g., buffered aqueous solution) containing same. According to these embodiments, a colorimetric change in the pH paper is indicative of a presence of the viral biomarker (e.g., 3CL^{pro} enzyme as described herein) in the sample. According to these embodiments, a colorimetric change in the pH paper is indicative of a presence of an active SARS-CoV-2 infection is a subject from the sample is drawn.

According to some of any of these embodiments, the method further comprises determining a level of the colorimetric change, so as to determine an amount of the viral biomarker in the sample, and thereby determine a level of an active viral infection (e.g., SARS-CoV-2) is a subject from the sample is drawn, in case of a biological sample. Determining the level of the colorimetric change can be performed as described herein in any of the respective embodiments.

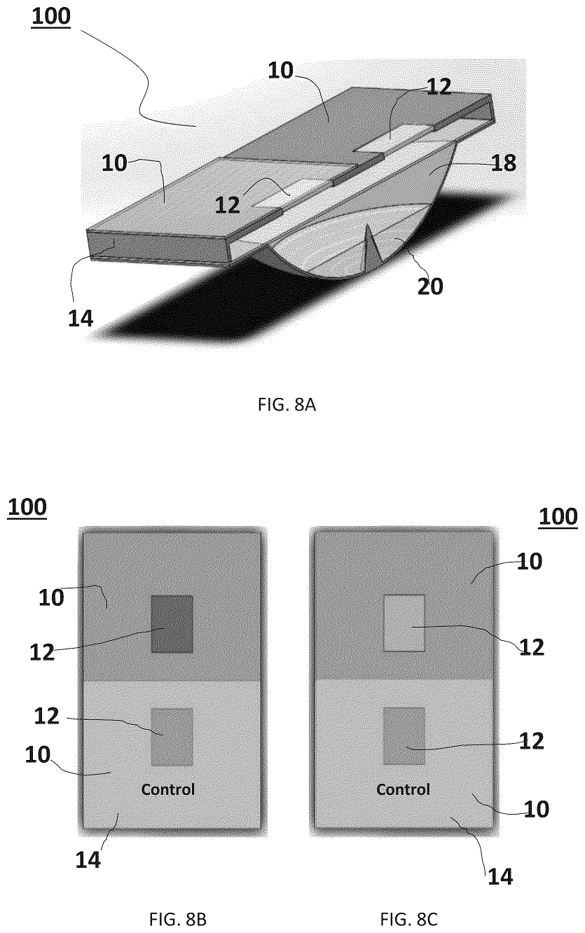

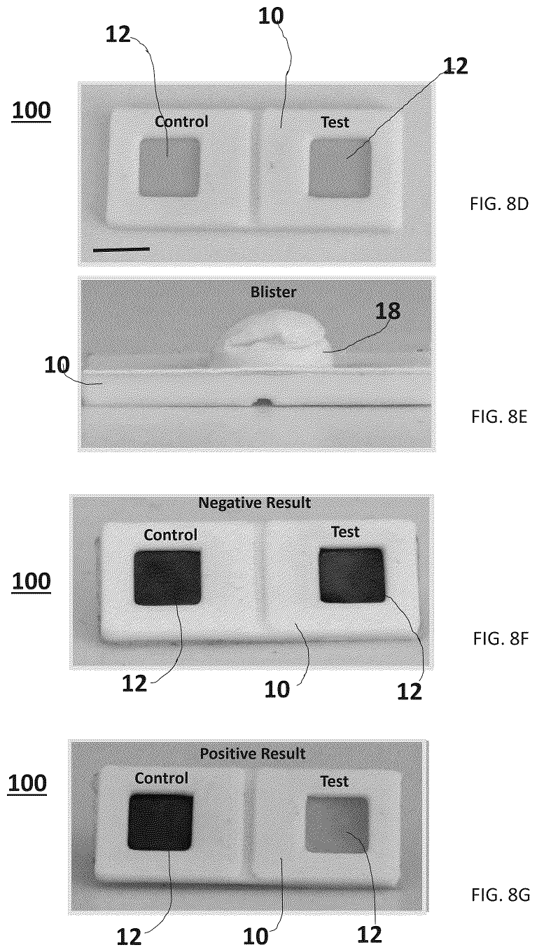

An exemplary method according to some of the present embodiments is schematically illustrated in FIG. 2A, and an exemplary testing strip for executing an exemplary method as described herein is presented in FIGS. 8A-C.

According to some embodiments of the present invention, a method as described herein is executed using a testing strip as described herein in any of the respective embodiments.

According to some embodiments of the present invention, a method as described herein is effected by contacting a saliva sample drawn from a subject with a testing strip as described herein.

According to some of any of the embodiments described herein, when a biological sample is a saliva sample, contacting the testing strip can be effected using dedicated means (e.g., a swab) or by spitting onto the strip (e.g., to a dedicated area of the strip).

According to some embodiments of the present invention, a method as described herein is effected by contacting a sample as described herein with an array of colorimetric indicators, as described herein for a multiplex system, so thereby determine a presence, level and/or type of a viral biomarker in a sample.

According to some embodiments of the present invention, a method as described herein is effected by contacting a sample as described herein with an array of colorimetric indicators, as described herein for a multiplex system, so thereby determine a presence, level and/or type of a viral infection in a biological sample.

According to an aspect of some embodiments of the invention, there is provided a composition or an article-of-manufacture or a composition-of-mater comprising a colorimetric indicator as described herein in any of the respective embodiments, which is or comprises a solid matrix as described herein in any of the respective embodiments, and an agent that selectively reacts with a viral biomarker as described herein in any of the respective embodiments, associated with the solid matrix.

According to some of any of the embodiments described herein, the selective agent (i.e., that selectively reacts with the viral biomarker as described herein in any of the respective embodiments) is absorbed to (in and/or on) the solid matrix as described herein in any of the respective embodiments.

Herein, the phrase “absorbed in and/or on the solid matrix” means an outcome of a process by which the agent that selectively reacts with the viral biomarker is taken up and retained within the internal structure of the solid matrix (absorbed in) and/or adheres to the surface of the solid matrix (absorbed on). This phrase encompasses both internal incorporation of the agent by the solid matrix and surface attachment of the agent that selectively reacts with the viral biomarker to the solid matrix material.

According to some of these embodiments, the solid matrix is or comprises a solid pH indicator, for example, a pH paper.

According to some of these embodiments, there is provided an article-of-manufacturing which comprises a pH paper having associated therewith (e.g., absorbed thereto; therein and/or thereon) an agent that selectively reacts with a pre-determined viral biomarker.

Articles-of-manufacturing as described herein can be stored while remaining stable and active for a time period as described herein.

According to some of any of the embodiments described herein, the composition or article-of-manufacturing further comprises an aqueous solution featuring a pH as described herein, optionally absorbed to the solid matrix (e.g., the pH paper).

Compositions or articles-of-manufacturing according to these embodiments can be prepared by contacting the solid matrix with a solution (e.g., an aqueous solution) comprising the selective agent. Optionally, the solid matrix is thereafter dried (e.g., air-dried or vacuum dried).

According to an aspect of some embodiments of the invention, there is provided a composition comprising an aqueous solution as described herein in any of the respective embodiments, and an agent that selectively reacts with a viral biomarker as described herein in any of the respective embodiments. Such a composition is also referred to herein as an indicator solution. Such a composition can be prepared simply by mixing the selective agent with an aqueous solution (e.g., buffered aqueous solution), as described herein in any of the respective embodiments. Optionally, the pH of the solution is pre-determined to differ from a pH that results from an interaction of the viral biomarker with the selective agent.

According to an aspect of some embodiments of the invention, there is provided a kit comprising an agent that selectively reacts with a viral biomarker as described herein in any of the respective embodiments, and a colorimetric indicator which is such that exhibits a colorimetric change as a result of a reaction between the agent and the viral biomarker as described herein in any of the respective embodiments.

According to some of any of the embodiments described herein, the kit comprises an aqueous solution that comprises the agent that selectively reacts with the viral biomarker (e.g., the aqueous solution that comprises the agent that selectively reacts with the viral biomarker as described herein in any of the respective embodiments), and the colorimetric indicator as described herein in any of the respective embodiments. In some of any of the embodiments described in this aspect, the colorimetric indicator and the aqueous solution are individually packaged in the kit.

According to some of any of the embodiments described herein, the kit comprises the (e.g., aqueous) solution that comprises the agent that selectively reacts with the viral biomarker and the (e.g., aqueous) solution that comprises the colorimetric indicator, as described herein in any of the respective embodiments. In some of any of the embodiments described in this aspect, the (e.g., aqueous) solution that comprises the colorimetric indicator and the (e.g., aqueous) solution that comprises the agent that selectively reacts with the viral biomarker are each individually packaged in the kit.

According to alternative embodiments, the aqueous solution that comprises the agent that selectively reacts with the viral biomarker further comprises the colorimetric indicator. In some of any of the embodiments described herein, the (e.g., aqueous) solution that comprises the agent that selectively reacts with the viral biomarker and the (e.g., aqueous) solution that comprises the colorimetric indicator are each individually packaged within the kit.

According to some of any of these embodiments, the kit further comprises means or a device (e.g., a swab) for collecting a biological sample (e.g. from a subject). According to some of any of these embodiments, the kit may further comprise instructions to contact the collected biological sample with the aqueous solution(s).

According to some of any of these embodiments, the colorimetric indicator comprises a solid matrix (e.g., a pH paper), and the kit comprises the solid matrix as described herein in any of the respective embodiments and any combination thereof. According to some of these embodiments, the pH paper has the selective agent associated therewith, as described herein. According to some of these embodiments, the kit further comprises an aqueous solution as described herein in any of the respective embodiments. Alternatively, the kit comprises a pH paper, and an aqueous solution that comprises the selective agent, as described herein in any of the respective embodiments and any combination thereof. In some of these embodiments, the pH paper and the aqueous solution are packaged individually within the kit. According to some of any of these embodiments, the kit may further comprise instructions to contact the collected biological sample with the pH paper and then contacting the pH paper with the aqueous solution(s). According to some of these embodiments, an expiry date of such a kit, or at least of the solid matrix, is at least one week, at least 2 weeks, at least 3 weeks, at least 4 weeks, or at least one month, from its manufacturing date.

According to some of any of these embodiments, the colorimetric indicator comprises a solid matrix (e.g., a pH paper), and the kit comprises the solid matrix as described herein in any of the respective embodiments and any combination thereof. According to some of these embodiments, the pH paper has an aqueous indicator solution as described herein associated with the pH paper. According to some of these embodiments, the kit further comprises an aqueous solution comprising the selective agent as described herein in any of the respective embodiments, individually packaged in the kit.

According to some of any of the embodiments described herein, the kit further comprises means or a device for collecting a biological sample from a subject.

According to some of any of the embodiments described herein, the colorimetric agent (e.g., in a form of a solid matrix) is mounted on or forms a part of the means or device.

According to some of any of the embodiments described herein, the kit is devoid of an antibody.

According to some of any of the embodiments described herein, the kit is for use in determining a presence and/or amount of a viral biomarker in a sample.

According to some of any of the embodiments described herein, the sample is a biological sample collected from a subject, and the kit is for use in determining a presence and/or level of a viral infection in the subject

According to some of any of the embodiments described herein, the kit comprises a test strip as described herein in any of the respective embodiments and any combination thereof. According to some of these embodiments, the kit or the test strip further comprises means or a device for collecting a biological sample from a subject.

According to an aspect of some embodiments of the present invention, there is provided a test strip (also referred to herein interchangeably as a testing strip) for determining a presence and/or level of a viral infection in a subject as described herein in any of the respective embodiments.

In some of any of the embodiments described herein, the test strip comprises at least one piece of a solid matrix as described herein in any of the respective embodiments and any combination thereof. According to some embodiments, the solid matrix (e.g., pH paper) has a selective agent associated therewith or absorbed thereto as described herein in any of the respective embodiments. According to some embodiments, the test strip further comprises a compartment which comprises an aqueous solution as described herein in any of the respective embodiments. According to some embodiments, the compartment is configured such that a fluid communication with the solid matrix (e.g., a pH paper) can be generated.

According to some of any of these embodiments, the test strip comprises at least two pieces of a solid matrix (e.g., a pH paper) as described herein in any of the respective embodiments. In some of these embodiments, the two pieces of pH paper are in fluid communication, such that upon contacting an aqueous solution with the test strip, both solid matrices are wetted to a substantially similar extent.

In some of any of the embodiments described herein, the test strip comprises at least two pieces of a solid matrix (e.g., pH paper) as described herein in any of the respective embodiments, and both matrices (e.g., pH papers) have the selective agent associated therewith or absorbed thereto as described herein in any of the respective embodiments. According to some of these embodiments, the test strip further comprises a compartment which comprises an aqueous solution as described herein in any of the respective embodiments. According to some of these embodiments, the compartment is configured such that a fluid communication with the at least two solid matrices (e.g., a pH papers) can be generated.

In alternative embodiments, the test strip comprises at least two pieces of a solid matrix (e.g., a pH paper) as described herein in any of the respective embodiments, and a compartment which comprises the (e.g., aqueous) solution that comprises the selective agent as described herein in any of the respective embodiments. According to some of these embodiments, the compartment is configured such that a fluid communication with the at least two solid matrices (e.g., a pH papers) can be generated.

With specific reference to the Figures, wherein same elements are identified by same numerals, FIG. 8A depicts an exemplary test strip according to the present embodiments. A test strip 100 comprises a plastic strip 10 with two pH paper windows 12, one is covered with a clear plastic 14 to serve as control window. Each of pH papers 12 has absorbed thereto (as described herein in any of the respective embodiments) an aqueous solution that comprises an agent that selectively reacts with a viral biomarker 16 as described herein in any of the respective embodiments (not shown; e.g., 3CL^{pro}-specific peptide substrate, e.g., SEQ ID NO: 2)), and is dried thereon (i.e., to provide two windows with pH papers 12 having absorbed thereto the agent that selectively reacts the viral biomarker 16 as described herein in any of the respective embodiments). In exemplary embodiments, test strip 100 further comprises a backside tank/blister 18 that comprises an aqueous solution 20 (as described herein in any of the respective embodiments; e.g., basic, e.g., pH=8.5).

FIG. 8C depicts an exemplary test strip according to the present embodiments. A test strip 100 comprises a plastic strip 10 with two pH paper windows 12, one is covered with a clear plastic 14 to serve as control window (not shown). Each of pH papers 12 has absorbed thereto (as described herein in any of the respective embodiments) an aqueous solution that comprises an agent that selectively reacts with a viral biomarker 16 as described herein in any of the respective embodiments (not shown; e.g., 3CL^{pro}-specific peptide substrate, e.g., SEQ ID NO: 2)), and is dried thereon (i.e., to provide two windows with pH papers 12 having absorbed thereto the agent that selectively reacts the viral biomarker 16 as described herein in any of the respective embodiments). In exemplary embodiments, shown in FIG. 8D, test strip 100 further comprises a backside blister 18 that comprises an aqueous solution 20 (as described herein in any of the respective embodiments; e.g., basic, e.g., pH=8.5).

According to an aspect of some embodiments of the present invention, there is provided a method for determining a presence and/or level of a viral infection in a subject using a test strip as described herein in any of the respective embodiments (e.g., test strip 100). The method, according to these embodiments, comprises: (i) contacting pH paper 12 which is not covered by clear plastic 14 with a sample (as described herein in any of the respective embodiments; e.g., a saliva sample), and (ii) contacting aqueous solution 20 with pH papers 12 (e.g., by breaking blister 18), by generating a fluid communication with pH papers 12, to thereby determine a presence and/or level of a viral biomarker in the sample, or a viral infection in a subject, in case a biological sample is used.

As shown in FIGS. 8B, 8C and 8D, upon contacting aqueous solution 20, a colorimetric change is observed in both pH papers 12. Within a time period of from 0.5 to 10, or 0.5 to 5, or 0.5 to 3, minutes, a presence and/or level of a viral biomarker is determined based on the presence or absence of a further color change, such that upon contacting aqueous solution 20, a presence of a viral biomarker is determined when pH paper 12 that was contacted with the sample has a further detectable colorimetric change; and an absence of a viral infection is determined when the two pH papers 12 do not exhibit a further colorimetric change following contacting aqueous solution 20.

A level of a colorimetric change and/or presence or absence thereof can be determined by converting a perceived color to a RGB signal as described herein in any of the respective embodiments (e.g., using a look-up table).

According to some of these embodiments, test strip 100 and optionally a means or a device (e.g., a swab) for collecting a biological sample (e.g. from a subject) are packaged together, optionally each individually, within a kit.

According to an aspect of some embodiments of the present invention, there is provided a colorimetric indicator-containing matrix. This colorimetric indicator-containing matrix (also referred to herein as a “system”) can be regarded as a multiplex system. In some of any of the embodiments of this aspect, the colorimetric indicator-containing matrix is usable in a multiplex method of detecting a presence, type, and/or amount of one or more viral biomarker/s in a sample (e.g., a biological sample) using a single system.

According to some of any of the embodiments of this aspect, the colorimetric indicator-containing matrix comprises a colorimetric indicator (e.g., as described herein in any of the respective embodiments), which is or comprises a solid matrix (e.g., the solid matrix as described herein in any of the respective embodiments; e.g., a pH paper), and a plurality of agents associated with the solid matrix (e.g., a first agent is attached to a first portion of the matrix, a second agent is attached to a second portion of the matrix, a third agent is attached to a third portion of the matrix, and so forth, whereby the agents are different from one another).

In some of any of the embodiments of this aspect, each of the agents specifically reacts with a different viral biomarker, each individually being as described herein in any of the respective embodiments.

In some embodiments, the system is such that each selective agent is absorbed to a different portion of the solid matrix of the colorimetric indicator.

In some embodiments, the system comprises a plurality of solid matrices, each having a different agent absorbed thereto, and the plurality of matrices forms a consolidated array.

The target viral biomarkers can be different biomarkers of the same virus or viral infection or such that each biomarker is indicative of a presence and/or amount of a different virus or viral infection, and the plurality of different agents are selected accordingly. Such matrices or systems allow testing (e.g., simultaneously) a single sample (e.g., biological sample) and determine a presence and/or level and/or type of a viral biomarker or a viral infection.

According to some of these embodiments, the system further comprises an aqueous solution, or a plurality of aqueous solutions, as described herein in any of the respective embodiments. Each aqueous solution is an aqueous solution as described herein in any of the respective embodiments.

According to some of any of the embodiments described herein, the system as described herein and the aqueous solution or the plurality of aqueous solution are packaged together, optionally each individually, within a kit.

In exemplary embodiments, each portion of the system has a different selective agent associated therewith (e.g., absorbed thein and/or thereon), and each selective agent is a substrate of a respective enzyme that is specific to a respective virus.

In exemplary embodiments, each portion of the system has a different selective agent associated therewith (e.g., absorbed thein and/or thereon), and each selective agent is a specific substrate of a proteolytic enzyme specific to a specific virus.

Exemplary viral specific enzymes are presented in Table B hereinabove.

According to an aspect of some embodiments of the present invention, there is provided a method of determining a presence, type, and/or amount of one or more viral biomarker/s in a sample (e.g., a biological sample), which is effected by contacting the sample with the multiplex system as described herein.

According to some embodiments of this method, the sample is contacted with the multiplex system, aqueous indicator solutions are contacted with the array of solid matrices, either the same solution, or each portion or each group of portion with a dedicated solution (which may differ from another, for example, by the pH), and the colorimetric change is detected as described herein in any of the respective embodiments and any combination thereof.

The method and system as described herein can be used in determining a presence and/or level and/or type of any of the viral biomarkers and the viral infections known in the art, for example, those presented in Tables A and B herein.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

The term “peptide” encompasses native peptide (e.g., degradation products, synthetically synthesized peptide and/or recombinant peptide), including, without limitation, native proteins, fragments of native proteins and homologs of native proteins and/or fragments thereof, as well as peptidomimetics (typically, synthetically synthesized peptide) and peptoids and semipeptoids which are peptide analogs, which may have, for example, modifications rendering the peptide more stable while in a body or more capable of penetrating into cells. Such modifications include, but are not limited to N-terminus modification, C-terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided herein below.

Peptide bonds (—CO—NH—) within the peptide may be substituted, for example, by N-methylated amide bonds (—N(CH₃)—CO—), ester bonds (—C(═O)—O—), ketomethylene bonds (—CO—CH₂—), sulfinylmethylene bonds (—S(═O)—CH₂—), α-aza bonds (—NH—N(R)—CO—), wherein R is any alkyl (e.g., methyl), amine bonds (—CH₂—NH—), sulfide bonds (—CH₂—S—), ethylene bonds (—CH₂—CH₂—), hydroxyethylene bonds (—CH(OH)—CH₂—), thioamide bonds (—CS—NH—), olefinic double bonds (—CH═CH—), fluorinated olefinic double bonds (—CF═CH—), retro amide bonds (—NH—CO—), peptide derivatives (—N(R)—CH₂—CO—), wherein R is the “normal” side chain, naturally present on the carbon atom.

These modifications can occur at any of the bonds along the peptide chain and even at several (e.g., 2, 3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptide of some embodiments of the invention may also include one or more modified amino acids or one or more non-amino acid monomers (e.g., fatty acids, complex carbohydrates etc.).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables C and D below list naturally occurring amino acids (Table C), and non-conventional or modified amino acids (e.g., synthetic, Table D) which can be used with some embodiments of the invention.

TABLE C

| Amino Acid | Three-Letter Abbreviation | One-letter Symbol |
|---|---|---|
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE D

| Non-conventional amino acid | Code | Non-conventional amino acid | Code |
|---|---|---|---|
| ornithine | Orn | hydroxyproline | Hyp |
| α-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) carbamylmethyl-glycine | Nnbhm |
| N-(3,3- | Nbhe | N-(N-(3,3-diphenylpropyl) | Nnbhe |
| diphenylpropyl)glycine | | carbamylmethyl-glycine | |
| 1-carboxy-1-(2,2-diphenyl | Nmbc | 1,2,3,4-tetrahydroisoquinoline-3- | Tic |
| ethylamino)cyclopropane | | carboxylic acid | |
| phosphoserine | pSer | phosphothreonine | pThr |
| phosphotyrosine | pTyr | O-methyl-tyrosine | |
| 2-aminoadipic acid | | hydroxylysine | |

The peptide of some embodiments of the invention are preferably utilized in a linear form, although it will be appreciated that in cases where cyclization does not severely interfere with peptide characteristics, cyclic forms of the peptide can also be utilized.

Since the present peptide are preferably utilized in therapeutics or diagnostics which require the peptide to be in soluble form, the peptide of some embodiments of the invention preferably include one or more non-natural or natural polar amino acids, including but not limited to serine and threonine which are capable of increasing peptide solubility due to their hydroxyl-containing side chain.

The peptide of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the invention involves solid phase peptide synthesis.

Large scale peptide synthesis is described by Andersson (2000) Biopolymers, 55(3), 227-250.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Experimental Methods

### Materials:

pH indicator paper (pH 6.4-8.0) was obtained from Macherey-Nagel, CAT: 030200.

3CL^{pro} enzyme (Recombinant derived from Escherichia coli, ab277614; SEQ ID NO: 1), HIV-2 Protease (Recombinant derived from Escherichia coli, ab84117; SEQ ID NO: 6), Human TMPRSS2 protein (Recombinant derived from Wheat germ, ab112364; SEQ ID NO: 3), and Chymotrypsin protein (Native human, ab90927; SEQ ID NO: 7) were obtained from ABCAM.

3CL^{pro} substrate (peptide, KTSAVLQSGFRKME; SEQ ID NO: 2) was obtained from Sigma-Aldrich™.

MERS-CoV 3CL Protease (Recombinant derived from Escherichia coli, E-719; SEQ ID NO: 4) and SARS-CoV 3CL Protease (Recombinant derived from Escherichia coli, E-718; SEQ ID NO: 5) were obtained from Novous biologicals.

Disodium hydrogen phosphate (S7907) was obtained from Sigma-Aldrich™.

Deionized water (18 MΩ·cm) were used throughout, unless otherwise mentioned.

Reaction indicator solution pH=8.5 (also referred to herein as “pH=8.5 solution”) was prepared from an aqueous solution of disodium hydrogen phosphate.

Imaging: Scanning electron microscopy (SEM) imaging was performed using Quanta 200 FEG ESEM, Thermo Scientific™, 20.0 kV, WD 10.0 mm.

Optical imaging was performed using Dino-Lite Edge 700-900× Digital USB Microscope 5MP or using a Samsung Galaxy™ Note10-lite cellphone camera.

Color processing: colors were processed using a standard processing tool. Specifically, images were analyzed using ‘Color Code Picker’ to provide the RGB, HEX and HSL color code of the pixel of a given image: www(dot)colorcodepicker(dot)com; however, any other color computational analysis is contemplated.

Antigen sample testing: COVID-19 antigen rapid saliva tests were obtained from GenSure™ (COVID-19 Antigen Rapid Test Kit P2004s). GenSure™ COVID-19 antigen test for saliva and nasal samples was used for self-testing by PCR-positive SARS-CoV-2 volunteers.

Clinical sample preparation and testing: The human saliva experiments were approved by the Ethics Committee of Tel Aviv University (0053-22-TLV). Saliva samples were collected in 15 ml sterile tubes and stored in refrigeration until measuring. No pretreatment steps were taken before measurements.

Kinetic measurements of absolute pH was calculated as follows:

$$Absolute pH = ( {pH}_{i}^{substrate} - {pH}_{t}^{substrate} ) - ( {pH}_{i}^{control} - {pH}_{t}^{control} )$$

pH was measured through pyranine fluorescence.

Sequences: Table 1 below presents the amino acid sequences of the peptide and proteins used herein.

TABLE 1

| | | | | SEQ |
|---|---|---|---|---|
| Construct | | | Mw | ID |
| name | Composition | Sequence | (kDa) | NO. |
| SARS-CoV-2 | Recombinant, | SGFR KMAF PSGK VEGC MVQV | 34 | 1 |
| 3CL^{pro} Enzyme | Escherichia coli- | TCGT TTLN GLWL DDVV YCPR HVIC | | |
| | derived | TSED MLNP NYED LLIR KSNH NFLV | | |
| | | QAGN VQLR VIGH SMQN CVLK | | |
| | | LKVD TANP KTPK YKFV RIQP GQTF | | |
| | | SVLA CYNG SPSG VYQC AMRP NFTI | | |
| | | KGSF LNGS CGSV GFNI DYDC VSFC | | |
| | | YMHH MELP TGVH AGTD LEGN | | |
| | | FYGP FVDR QTAQ AAGT DTTI TVNV | | |
| | | LAWL YAAV INGD RWFL NRFT TTLN | | |
| | | DFNL VAMK YNYE PLTQ DHVD ILGP | | |
| | | LSAQ TGIA VLDM CASL KELL QNGM | | |
| | | NGRT ILGS ALLE DEFT PFDV VRQC | | |
| | | SGVT FQ | | |
| SARS-CoV-2 | Peptide | KTSA VLQS GFRK ME | 1.6 | 2 |
| 3CL^{pro} | | | | |
| Substrate | | | | |
| Human | Recombinant, | GWGA TEEK GKTS EVLN AAKV LLIE | 38 | 3 |
| TMPRSS2 | Wheat germ- | TQRC NSRY VYDN LITP AMIC AGFL | | |
| | derived | QGNV DSCQ GDSG GPLV TSKN | | |
| | | NIWW LIGD TSWG SGCA KAYR | | |
| | | PGVY GNVM VFTD WIYR QMRA DG | | |
| MERS-CoV | Recombinant, | SGLV KMSH PSGD VEAC MVQV | 34 | 4 |
| 3CL Protease | Escherichia coli- | TCGS MTLN GLWL DNTV WCPR | | |
| | derived | HVMC PADQ LSDP NYDA LLIS MINH | | |
| | | SFSV QKHI GAPA NLRV VGHA MQGT | | |
| | | LLKL TVDV ANPS TPAY TFTT VKPG | | |
| | | AAFS VLAC YNGR PTGT FTVV MRPN | | |
| | | YTIK GSFL CGSC GSVG YTKE GSVI | | |
| | | NFCY MHQM ELAN GTHT GSAF | | |
| | | DGTM YGAF MDKQ VHQV QLTD | | |
| | | KYCS VNVV AWLY AAIL NGCA | | |
| | | WFVK PNRT SVVS FNEW ALAN QFTE | | |
| | | FVGT QSVD MLAV KTGV AIEQ LLYA | | |
| | | IQQL YTGF QGKQ ILGS TMLE DEFT | | |
| | | PEDV NMQI MGVV MQ | | |
| SARS-CoV | Escherichia coli- | SGFR KMAF PSGK VEGC MVQV | 34 | 5 |
| 3CL Protease | derived | TCGT TTLN GLWL DDTV YCPR HVIC | | |
| | | TAED MLNP NYED LLIR KSNH SFLV | | |
| | | QAGN VQLR VIGH SMQN CLLR | | |
| | | LKVD TSNP KTPK YKFV RIQP GQTF | | |
| | | SVLA CYNG SPSG VYQC AMRP NHTI | | |
| | | KGSF LNGS CGSV GFNI DYDC VSFC | | |
| | | YMHH MELP TGVH AGTD LEGK | | |
| | | FYGP FVDR QTAQ AAGT DTTI TLNV | | |
| | | LAWL YAAV INGD RWFL NRFT TTLN | | |
| | | DFNL VAMK YNYE PLTQ DHVD ILGP | | |
| | | LSAQ TGIA VLDM CAAL KELL | | |
| | | QNGM NGRT ILGS TILE DEFT PFDV | | |
| | | VRQC SGVT FQ | | |
| HIV-2 Protease | Escherichia coli- | PQFS LWKR PVVT AHIE GQPV EVLL | 11 | 6 |
| | derived | DTGA DSIV AGIE LGSN YSPK IVGG | | |
| | | IGGF INTK EYKN VEIE VLNK RVRA | | |
| | | TIM TGDT PINI FGRN ILAS GMSL NL | | |
| Chymotrypsin | Native human | MLGITVLAALLACASSC | 25 | 7 |
| | Chymotrypsin | GVPSFPPNLSARVVGGE | | |
| | protein | DARPHSWPWQISLQYL | | |
| | | KNDTWRHTCGGTLIAS | | |
| | | NFVLTAAHCISNTRTYR | | |
| | | VAVGKNNLEVEDEEGS | | |
| | | LFVGVDTIHVHKRWNA | | |
| | | LLLRNDIALIKLAEHVE | | |
| | | LSDTIQVACLPEKDSLL | | |
| | | PKDYPCYVTGWGRLW | | |
| | | TNGPIADKLQQGLQPV | | |
| | | VDHATCSRIDWWGFR | | |
| | | VKKTMVCAGGDGVIS | | |
| | | ACNGDSGGPLNCQLEN | | |
| | | GSWEVFGIVSFGSRRGC | | |
| | | NTRKKPVVYTRVSAYID | | |
| | | WINEKMQL | | |

(Table 1; Cont.)

### Example 1

### Sensor Design and Mode of Action

3CL^{pro} (SEQ ID NO: 1) is a cysteine protease; its catalytic site holds a catalytic dyad of Cys145-His41, and it usually cleaves at the LeuGln*Ser pattern (the cleaving site is marked with *) [Hoffman et al., 2020, supra]. Serine could be replaced with either Ala or Gly [Zhang et al., 2020, supra; and Senger et al. Mem. Inst. Oswaldo Cruz 2020, 115]. A well-known nucleophilic reaction catalyzes the peptide bond hydrolysis:
(a) Deprotonation of the catalytic dyad's Cys thiol by the His residue. The anionic sulfur initiates a nucleophilic attack of the substrate's carbonyl carbon, followed by the protonation of the bond's N-terminus by the His residue of the catalytic site, leading to its detachment from the substrate.
(b) The thioester intermediate formed by the C-terminus of the substrate and Cys residue is then hydrolyzed to regenerate the catalytic site and produce a carboxylic acid, causing an in-vitro pH drop in a non-buffered medium [Wang et al. ACS Catal. 2020, 10 (10), 5871-5890; and Huang et al. Biochemistry 2004, 43 (15), 4568-4574].

It was previously reported (Borberg et al., 2022, supra) that the enzymatic activity turnover rate acts as an amplifier to the diagnostic signal since one protease molecule can hydrolyze substrate molecules at a rate of about 60 molecules/minute [Zhu et al., 2020, supra; and Jin et al., 2020, supra]. Therefore, a 3CL^{pro}-specific peptide substrate (see, Table 1; SEQ ID NO:2; Chan et al., 2021, supra) was utilized for its high affinity and turnover rate for SARS-CoV-2 3CL^{pro} (SEQ ID NO:1).

Since 3CL^{pro} tends to dimerize, and the turnover rate of the tested 3CL^{pro} substrate (SEQ ID NO: 2) is about 60 substrate molecules per minute, the activity of 3CL^{pro} could be calculated by predicting that 80 pmol 3CL^{pro} (SEQ ID NO: 1) would cleave 1.2-2.4 nmol of the substrate in 120 seconds, for 80 μl well:

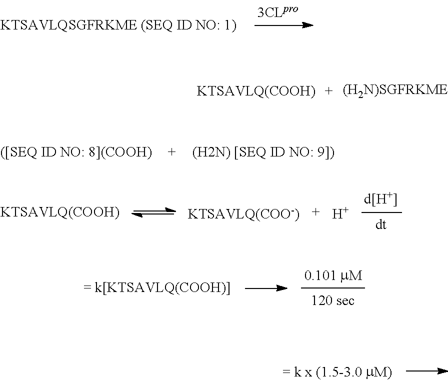

$$k = ( 2.8 - 5.6 ) x {10}^{- 4} \frac{1}{sec}$$

For 900 μl cell with the same presence of 3CL^{pro}, and starting pH is 7.40:

$$\frac{d [ {H}^{+} ]}{d t} = ( 2.8 - 5.6 ) x {10}^{- 4} \frac{1}{sec} \times ( 0.13 - 0.27 μM ) \to d [ {H}^{+} ] = 0.05 - 0.18 μM$$

$${[ {H}^{+} ]}_{f} = 0.09 - 0.22 μM \to pH = 6.66 - 7.05 \to Δ pH = 0.35 - 0.74$$

The present inventors have conceived employing the decrease in pH following the enzymatic activity of 3CL^{pro} (SEQ ID NO: 1), for detecting the presence of 3CL^{pro} and hence the presence of SARS-CoV-2 in a biological sample by colorimetry, using a pH indicator (specifically, a pH indicator paper, herein also a pH paper). A library of substrates has been recognized for 3CL^{pro} (see, for example, SEQ ID NOs: 2 and 10-30); the 3CL^{pro} substrate used in the Examples herein (SEQ ID NO: 2) showed a high affinity and turnover rate [Chan et al., Discovery of SARS-CoV-2 M^{pro} Peptide Inhibitors from Modelling Substrate and Ligand Binding, Chem. Sci. 2021, 12, 13686-13703; Grum-Tokars et al., Virus Res 2008, 133, 63; El-Baba et al., Angewandte Chemie International Edition 2020, 59, 23544; and Hoffman et al., J. Med. Chem. 2020, 63, 12725].

While reducing the present invention to practice, the present inventors have designed and successfully practiced a colorimetric detection methodology for determining a presence and optionally an amount of the presence of 3CL^{pro} (SEQ ID NO: 1) in a biological sample. While this method has been exemplified herein for the detection of the presence of 3CL^{pro} (SEQ ID NO: 1) as a viral biomarker, it can be readily adopted for detecting any viral biomarker that generates a pH change as a result of a viral infection.

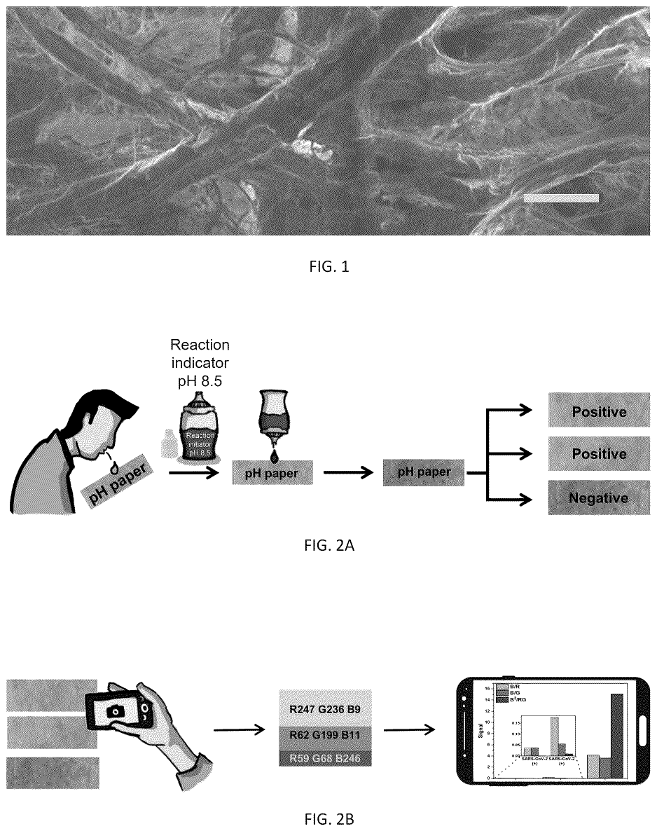

Colorimetric detection can be achieved using a commercially available pH indicator paper. Scanning electron microscopy (SEM) images of the paper fibers of an exemplary commercially available pH indicator paper were captured and are shown in FIG. 1. It is assumed that the paper fibers serve as a 3D sieve for biomolecules, including 3CL^{pro} (SEQ ID NO: 1) in the case of SARS-CoV-2-positive patient.

An exemplary devised colorimetry detection methodology is as follows:
(i) a pH indicator paper is contacted with a liquid biological sample in which a viral biomarker is present in case of a viral infection (e.g., saliva sample; blood sample) to thereby physically absorb biomolecules onto the paper's surface;
(ii) a buffered indicator solution containing a viral biomarker-specific agent, as described herein, is contacted with the pH paper to which the biological sample is absorbed;
(iii) a change in the paper's color, as a result of pH change compared to the indicator solution is then monitored, whereby a change in the paper's color is indicative of a presence of the biological marker in the sample.

An exemplary method of detecting a presence of 3CL^{pro} (SEQ ID NO: 1) as indicative of the presence of SARS-CoV-2 in a biological sample is as follows:
(a) An untreated saliva sample (3 μl) is dripped onto a pH indicator paper to thereby allow biomolecules (including 3CL^{pro} (SEQ ID NO: 1) molecules in a SARS-CoV-2-positive patient) to physically adsorb onto the surface of the paper; and
(b) A pH 8.5 buffer solution containing a 3CL^{pro}-specific peptide substrate (8 μl; SEQ ID NO: 2) is dripped onto the pH indicator paper.

In the presence of 3CL^{pro} (SEQ ID NO: 1) in the sample, a rapid pH drop occurs, which is monitored by a rapid change in color of the pH paper.

Without being bound to any particular theory, it is assumed that once the solution containing a 3CL^{pro}-specific peptide substrate (e.g., SEQ ID NO: 2) is dripped onto a pH indicator paper with a SARS-CoV-2-positive sample (i.e., including 3CL^{pro} (SEQ ID NO: 1) molecules), it results in a reaction between the surface-adsorbed 3CL^{pro} (SEQ ID NO: 1) molecules and the 3CL^{pro}-specific peptide substrate (e.g., SEQ ID NO: 2), which in turn results in hydrolysis of the peptide substrate by the protease and formation of acidic products as described herein. The diagnostic signal (i.e., formation of acidic products) is amplified by about 120-fold within 2 minutes, and is visualized by the decrease in pH. Thus, the pH indicator paper operates both as a 3D sieve sample collector and as a detection reporter for negative (more basic, blue) or positive (more acidic, yellow-green) enzymatic activity by a viral protease, as illustrated in FIG. 2A.

An optical camera can then be used to photograph the pH indicator paper and detect the change in pH, and the color of the image can be extrapolated into red-green-blue (RGB) values using a standard color analyses tool, e.g., Image Color Picker [Moroney et al., “Robust Color Extrapolation with Median Matrices”, 23rd Color and Imaging Conference Final Program and Proceedings, 2015, 171-174].

An optical readout of exemplary results is schematically illustrated in FIG. 2B. Such an optical processing allows POC untrained testers to use a mobile phone to take a photo of the pH indicator paper. The image is then processed into RGB values that are translated into diagnostic positive/negative results in a tool such as a cellphone application. Subsequently, results can be securely shared via mobile phone with healthcare providers and/or authorities, allowing at-home care and effortless epidemic management. The integration aids in overcoming the qualitative or semi-quantitative nature of paper-based biosensors [Antiochia, 2021, supra].

### Example 2

### Color Reaction Characterization and Color Extrapolation

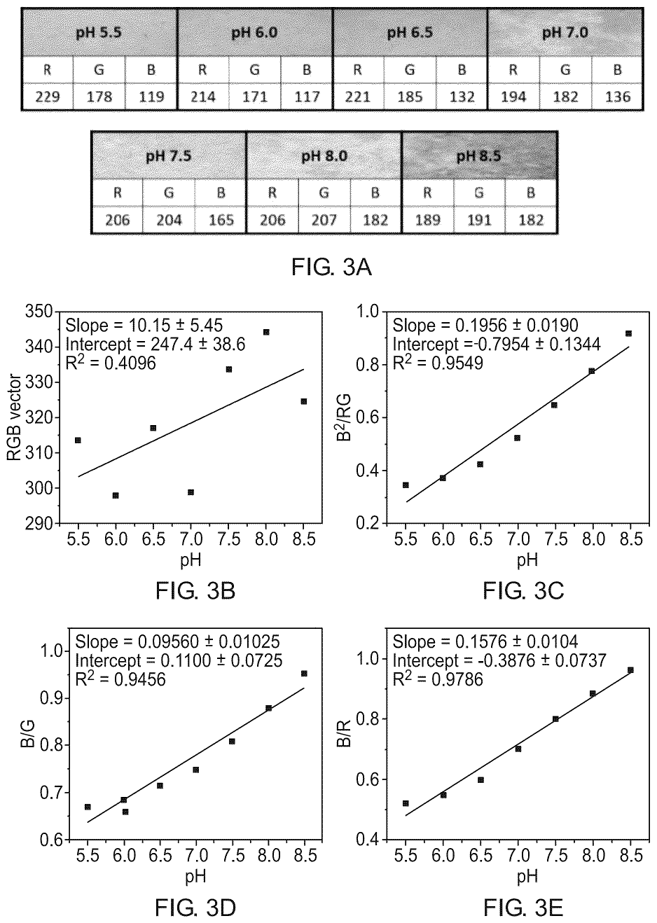

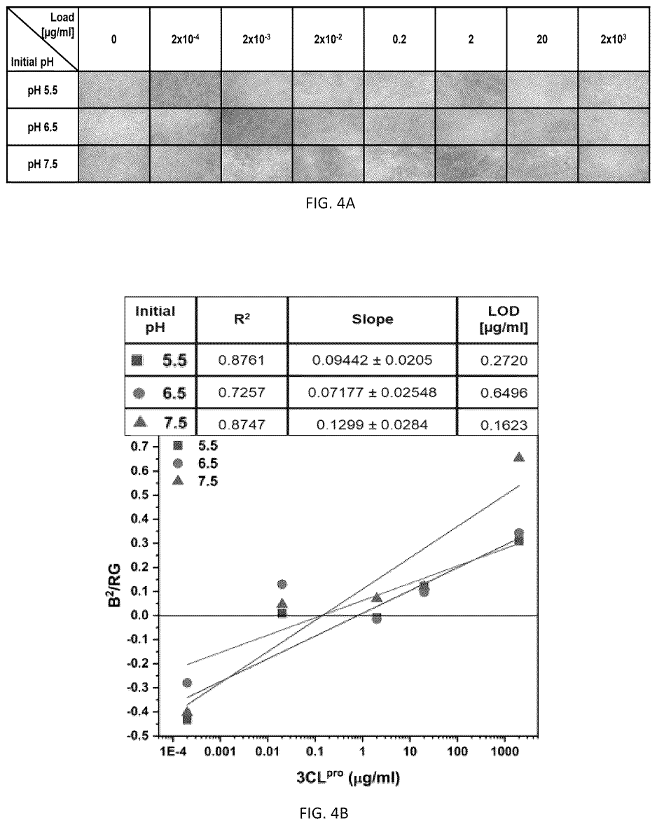

The colorimetric reactions of the pH paper following exposure to solutions of different pH values were photographed. Image Color Picker was used to analyze the photographs and extrapolate RGB values for each tested pH value. The results are summarized in FIG. 3A, wherein R is the extrapolated red coordinate value, G is the extrapolated green coordinate value, and B is the extrapolated blue coordinate value.

Then, at varying pH levels, the following RGB-derived values were fitted:

$$R B G vector = \sqrt{{R}^{2} + {G}^{2} + {B}^{2}} \qquad ( 1 )$$

$${B}^{2} / R G \qquad ( 2 )$$

$$B / G \qquad ( 3 )$$

$$B / R \qquad ( 4 )$$

Linear fitting of these RGB-derived values as a function of pH are presented in FIGS. 3B-E, and the fittings parameters of each signal are also indicated.

It can be seen that B²/RG, B/G and B/R (FIGS. 3C-E, respectively) demonstrate a linear correlation to the pH of the solution with high R² values. FIG. 3B indicates that the RGB vector is non-linear to pH, which may be due to the difference in image brightness. Therefore, in further experiments, RGB values were converted to signals referring to the blue coordinate value relative to the green (B/G) or red coordinates values (B/R), and to B²/RG.

### Example 3

### Optimization

In order to examine the response of the pH indicator paper to different conditions, the influence of different enzyme concentrations was examined.

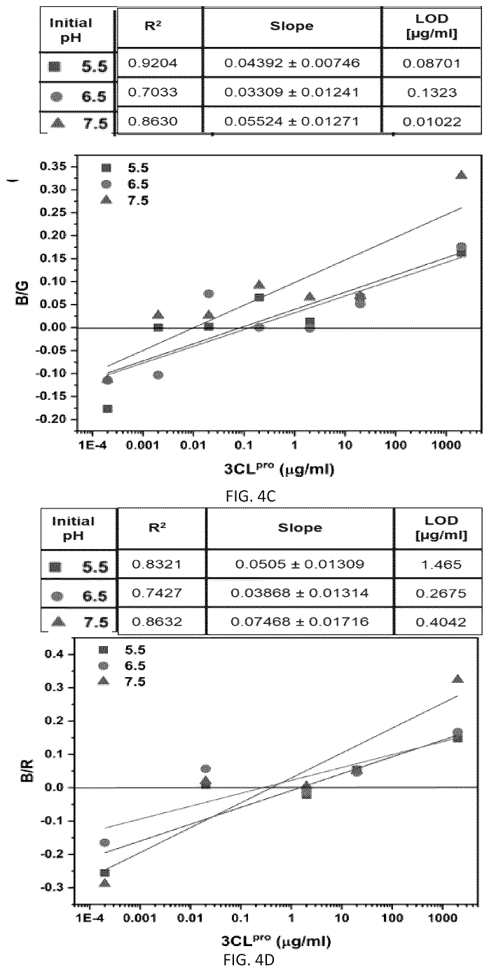

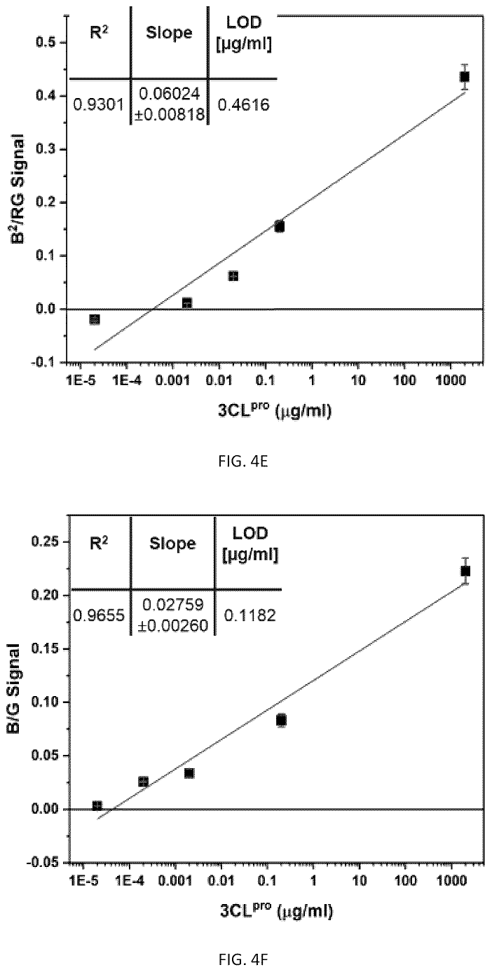

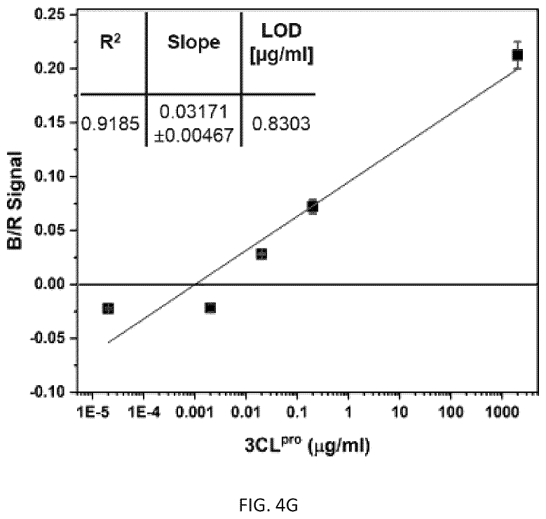

The colorimetric reaction of the pH indicator paper to healthy saliva samples was spiked with different 3CL^{pro} (SEQ ID NO: 1) concentrations with initial pH values of 5.5, 6.5, and 7.5. The results are presented in FIG. 4A and the extrapolated values and signals are presented in FIGS. 4B-G.

Due to the uniform starting point created by the reference solution (pH=8.5) containing the 3CL^{pro}-specific substrate (SEQ ID NO: 2), the change in pH is consistent within the viral 3CL^{pro} (SEQ ID NO: 1) enzymatic load, regardless of the starting pH value of the enzyme-spiked solution (FIGS. 4A-D).

Based on the obtained values (Valueᵢ, as seen in FIG. 4A) and relatively to the values given by a viral load of 0 μg/ml 3CL^{pro} (SEQ ID NO: 1) (Value₀), the RGB-derived signal was calculated for each B²/RG, B/G and B/R, as follows:

$$Signal = {Value}_{0} - {Value}_{i} \qquad ( 5 )$$

The RGB-derived signals were analyzed and plotted as a function of 3CL^{pro} (SEQ ID NO: 1) concentration, as depicted in FIGS. 4E-G.

A clear linear correlation is found between the RGB-derived signals to the concentration of 3CL^{pro} (SEQ ID NO: 1) in saliva. The best correlation was achieved by analyzing the B/G signal (FIG. 4F), which exhibits a high fitting coefficient, R²=0.9655, and a limit of detection (LOD) of 0.12 μg ml⁻¹; therefore, the B/G signal was chosen for further clinical detection of SARS-CoV-2.

### Example 4

### Specificity of the Platform

In order to further examine the response of the pH indicator paper that has the SARS-CoV-2 3CL^{pro} substrate (SEQ ID NO: 2) absorbed thereto to different conditions, analysis of samples containing different proteolytic enzymes was tested.

The colorimetric response of the pH indicator paper to healthy saliva samples spiked with different types of viral or human proteases was measured.

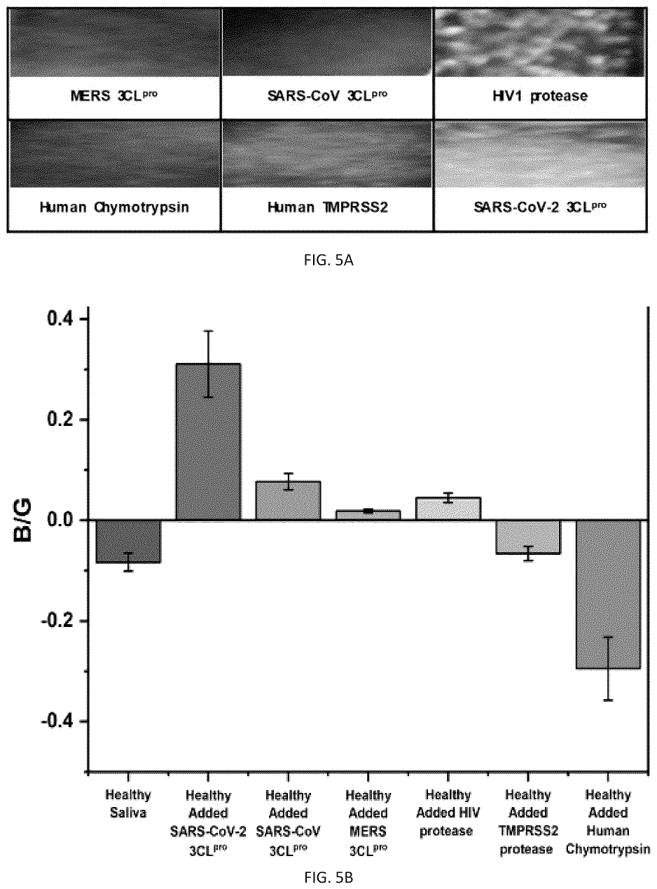

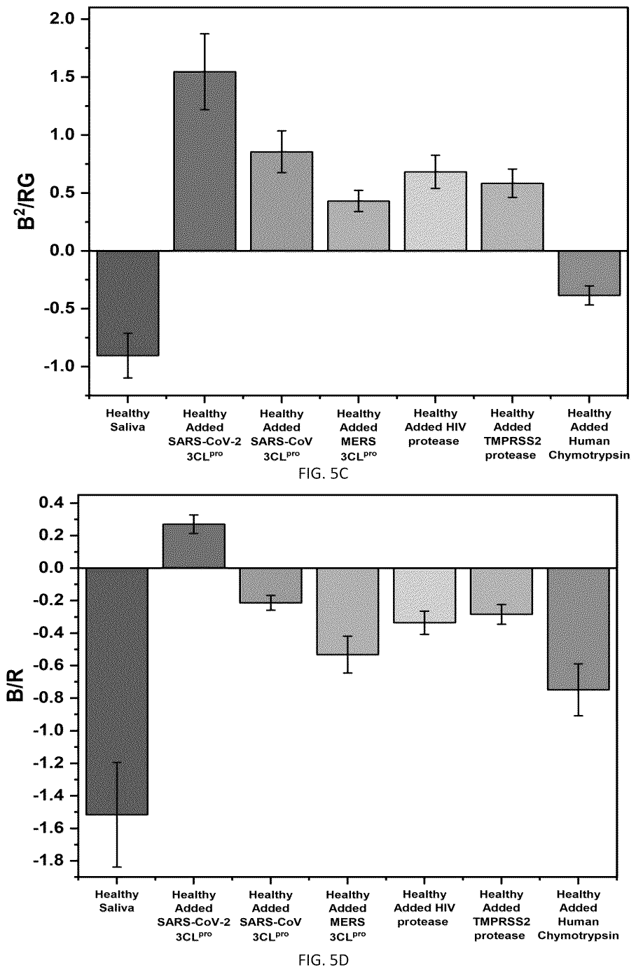

For this purpose, potentially cross-reacting proteases were chosen: 3CL^{pro} originating from severe acute respiratory syndrome coronavirus (SARS-CoV; SEQ ID NO: 5) and Middle East respiratory syndrome coronavirus (MERS-CoV; SEQ ID NO: 4), human immunodeficiency virus protease (HIV-2 Protease; SEQ ID NO: 6), and the human proteases TMPRSS2 (SEQ ID NO: 3) and Chymotrypsin (SEQ ID NO: 7) have been tested in comparison with the SARS-CoV-2 3CL protease (SEQ ID NO: 1), and the optical images are presented in FIG. 5A.

No detectable responses were observed for any of the tested proteolytic enzymes. The tested proteases resulted in B/G values lower than the detection threshold, as can be seen from FIG. 5B. Similarly, B²/RG and B/R values in response to the different proteases were also bellow the testing threshold, as shown in FIGS. 5C-D.

Considering the high similarity between 3CL^{pro} originating from SARS-CoV, MERS-CoV, and SARS-CoV-2 (SEQ ID NOs: 5, 4 and 1, respectively), the data shown in FIGS. 5A-B are unexpected.

Since the peptide substrate (e.g., SEQ ID NO: 2) has the potential to be cleaved by all the tested proteolytic enzymes, it was hypothesized that the observed difference in pH value is due to the lower amount of substrate molecules cleaved at the two-minute cutoff in which measurement is taken.

In order to corroborate this estimation, a series of kinetic measurements was conducted to measure the change in absolute pH following exposure of the peptide substrate (SEQ ID NO: 2) to 3CL proteases from SARS-CoV, MERS-CoV, and SARS-CoV-2 (SEQ ID NOs: 5, 4 and 1, respectively), wherein absolute pH at t minutes was calculated as follows:

$$Absolute pH = ( {pH}_{i}^{substrate} - {pH}_{t}^{substrate} ) - ( {pH}_{i}^{control} - {pH}_{t}^{control} ) \qquad ( 6 )$$

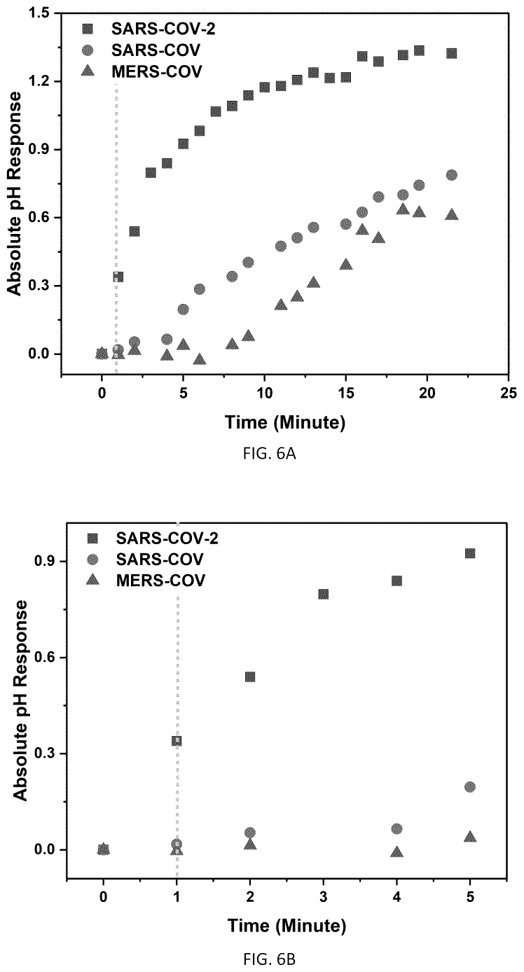

Full 22 minutes kinetics (FIG. 6A) and a 5-minute close-up (FIG. 6B) show the distinct difference in turnover rate between the three proteases.

These data indicate that although all the tested proteases can cleave the peptide substrate (SEQ ID NO: 2) to a given extent, the pH change caused by the presence of SARS-CoV-2 within two minutes is about 10 times higher than that of 3CL^{pro} from SARS-CoV and MERS-CoV, which allows its detection by the pH-paper platform.

These data show the surprisingly high intrinsic specificity of this platform against potential interference, which may be due to the high turnover rate for the hydrolysis of the substrate by 3CL^{pro} originating from SARS-CoV-2 (SEQ ID NO: 1).

Altogether, this detection platform showed low cross-reactivity with expected interferences, including presence of a virus of the same family (e.g., coronaviridae).

[symbol]

### Example 5

### Specificity and Sensitivity

To examine the detection ability of SARS-CoV-2 in clinical samples, a set of blind testing of forty-six volunteer subjects was performed.

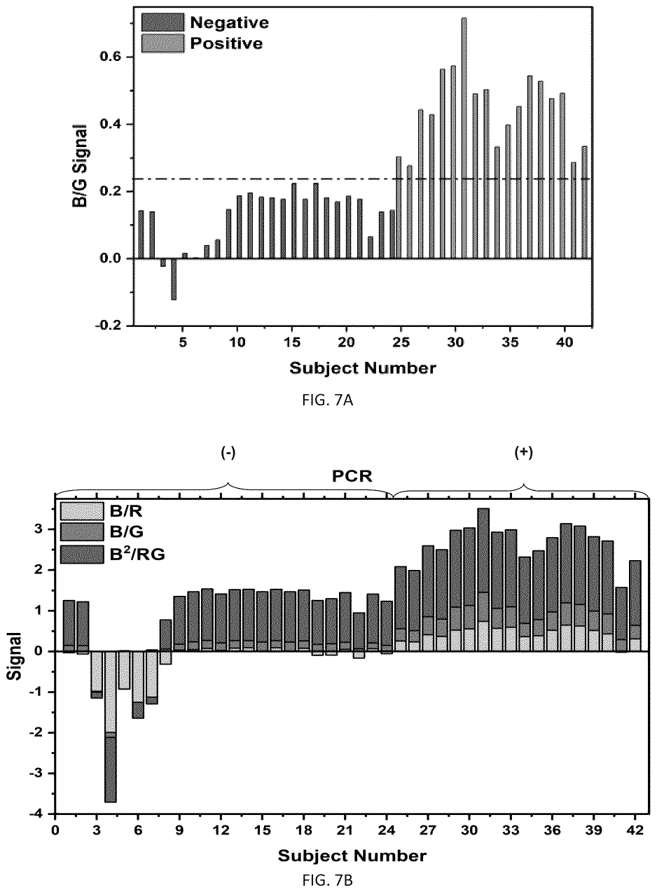

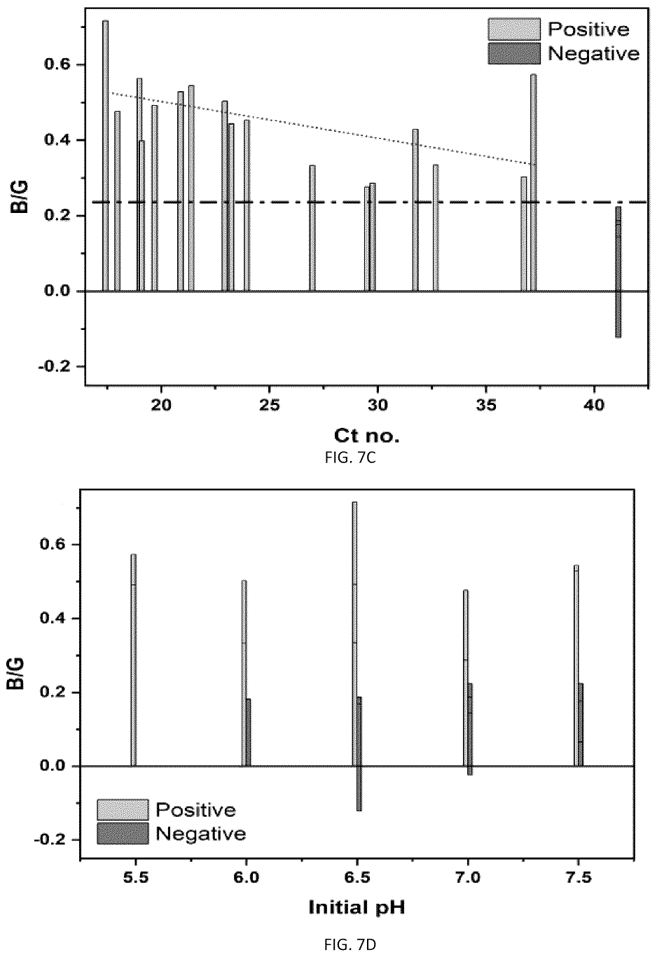

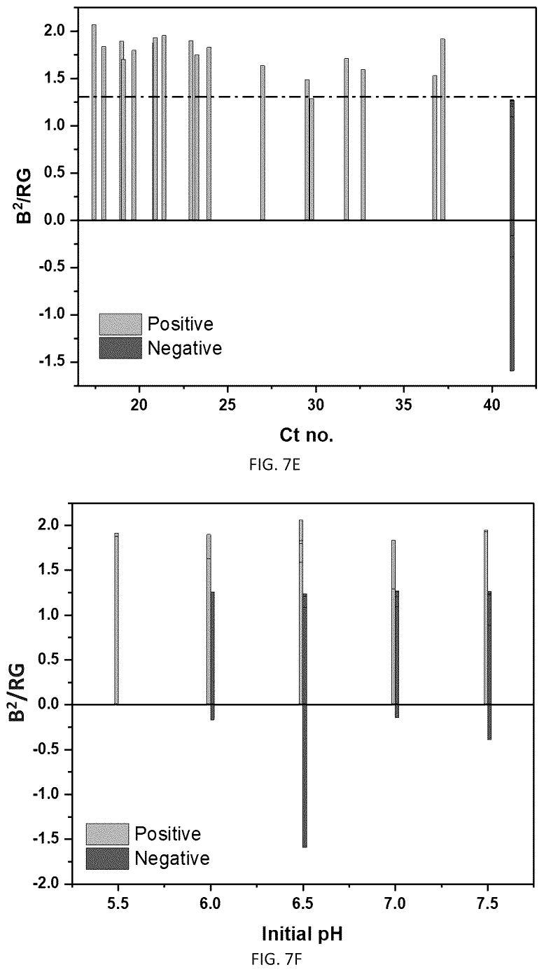

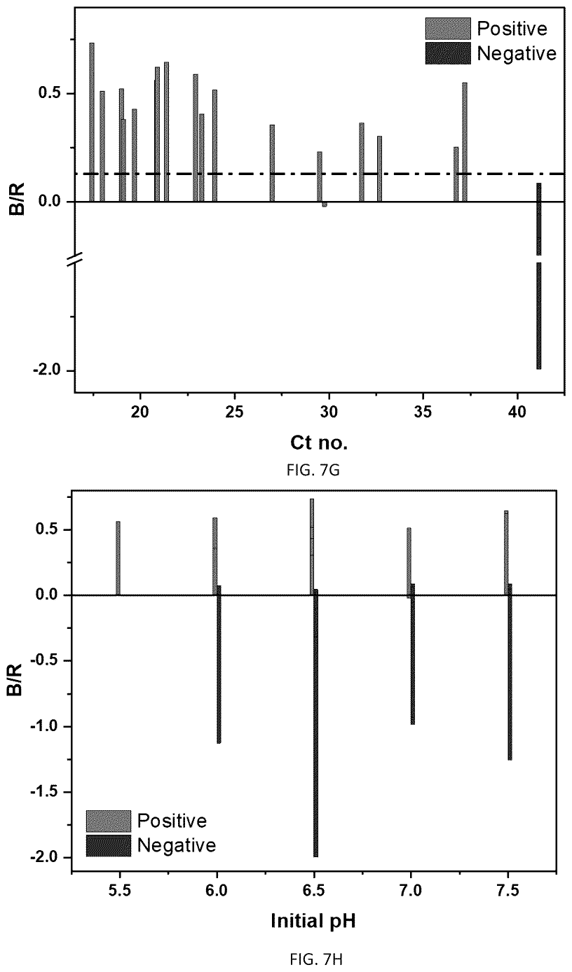

The pool of subjects included twenty-four SARS-CoV-2 negative samples (i.e., healthy) and eighteen SARS-CoV-2 positive samples (PCR-positive; having cycle threshold (Ct) value in the range of 17<Ct<40) of male and female volunteers. FIG. 7A shows the average of B/G signals per subject, and FIG. 7B shows the average of B/G, B/R and B²/RG signals per subject.

All SARS-CoV-2 positive subjects have been positively flagged for SARS-CoV-2 infection and were easily differentiated from healthy subjects, with a cutoff value of 0.24, since the lowest B/G signal of SARS-CoV-2-positive subjects was about 0.28, while the highest B/G signal of healthy subjects was about 0.22.

These results correlate to specificity and sensitivity values, as summarized in Table 2:

TABLE 2

| | B/G | | B²/RG | | B/R | |
|---|---|---|---|---|---|---|
| | Positive | Negative | Positive | Negative | Positive | Negative |
| PCR-positive subjects (#) | 18 | 0 | 17 | 1 | 17 | 1 |
| PCR-negative subjects (#) | 0 | 24 | 0 | 24 | 0 | 24 |

| | | | |
|---|---|---|---|
| Specificity | 100% | 100% | 100% |
| Sensitivity | 100% | 94% | 94% |

As can be seen, examination of B/G signal indicate 100% specificity and up to 100% sensitivity. The signals for B²/RG and B/R provided 100% specificity and 94% sensitivity.

In order to examine how the detection method is affected by the PCR Ct values and by the biological sample's initial pH, the data were plotted for each subject as a function of the obtained B/G signal, and are presented in FIGS. 7C and 7D, respectively. The data for B²/RG and B/R signals results are presented in FIGS. 7E-F and 7G-H, respectively.

In FIG. 7C, the blue dotted line indicates a trend of stronger signals with lower Ct values, meaning a stronger signal for higher viral loads. This is not the case when plotting B/G signal as a function of samples' initial pH, as shown in FIG. 7D; discrimination between positive and negative subjects is persistent regardless of the samples' initial pH, and no apparent trend could be found. Therefore, as suggested by earlier findings, the initial pH of the biological samples (saliva) does not affect the result of the detection method.

To summarize, in blind testing of forty-two clinical saliva samples, the platform demonstrated sensitivity and specificity of the method at 100% when focusing on B/G signals, thus demonstrating its high reliability.

Taken together, these data suggest that the RGB-derived signal from the detection platform is caused by the viral biomarker (3CL^{pro} enzymatic activity in a replicating virus), and is unrelated to the initial pH of the biological sample. As data suggests that the detection method is indeed based on 3CL^{pro} enzymatic activity as a viral biomarker, it is potentially more reliable than detection methods based on RNA as a biomarker, as detecting RNA may give false-positive results by detecting viral RNA fragments residues long after the viral replication is no longer active [Wu et al., BMC Medicine 2021, 19 (1), 77].

### Example 6

### At-Home Virus Detection Test Model

To demonstrate the applicability of the novel virus detection method, a testing strip was modeled.

As illustrated in FIGS. 8A-C, a test model 100 includes a plastic strip 10 with two pH indicator paper windows 12, one is covered with a clear plastic 14 to serve as control window and the second one is embedded with a dried solution of the 3CL^{pro}-specific enzymatic peptide substrate 16 (not shown) in an amount of 1-10 μgram. A backside tank/blister 18, which is visible in FIGS. 8A and 8E, contained a pH=8.5 solution 20.

A low-cost prototype device as herein described was constructed, as shown in FIGS. 8D, 8F and 8G (top view before (FIG. 8D) and after (FIGS. 8F and 8G) contacting a test sample, and in FIG. 8E (a side view), and preliminary tested.

The exposed substrate-embedded pH indicator paper window served as the test window, and the covered pH paper window served as the control window.

Buccal swabbing in multiple tested subjects indicated an average of 10-20 μl of native saliva was adsorbed by the 8 mm×8 mm test-paper slice. After saliva swabbing on test window 12, bottom blister 18 is popped to allow contact between the pH papers and pH=8.5 solution 20, and both pH paper in the windows 12 turned blue. When the tested subject is COVID-19 negative, both windows remain blue, as illustrated in FIG. 8B. If the subject is COVID-19 positive, the test window turns yellow-green, as illustrated in FIG. 8C.

Indeed, as can be seen in FIGS. 8G and 8F, negative and positive results were obtained for COVID-19-negative and COVID-19-positive subjects, respectively, when using the detection platform. The complete sample-to-test time was less than one minute.

By comparing the B/G signals of the two paper windows, as described herein, the output of the test is easily read via a mobile phone app and/or a website, which can also transfer test results to a health provider and/or authority, as illustrated in FIG. 2B.

### Example 7

### Stability

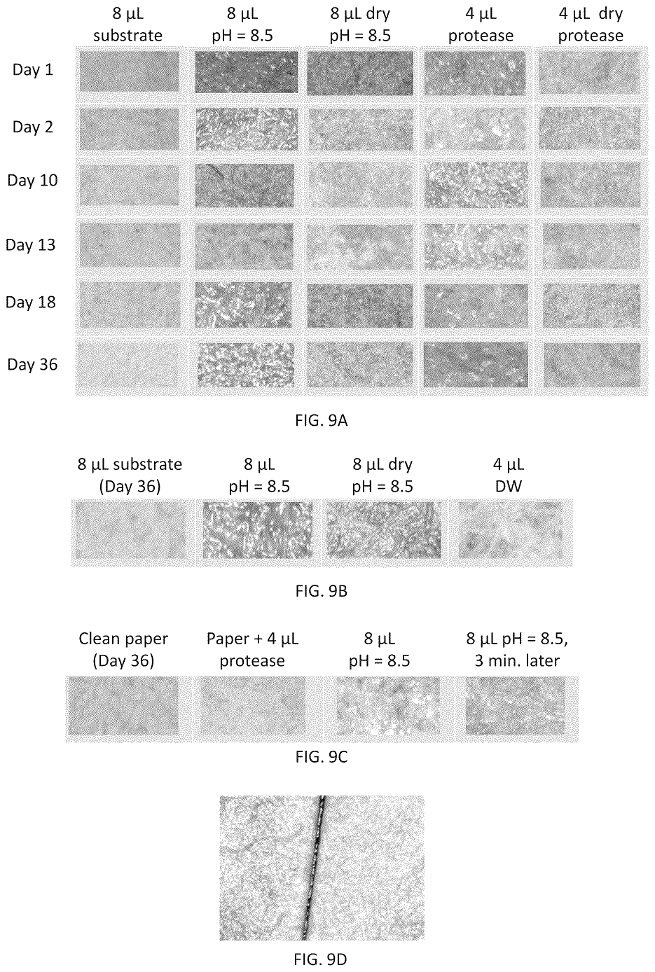

The stability of a pH paper-based platform as described herein (e.g., in Example 6) was tested, and the results are presented in FIGS. 9A-D.

FIG. 9A shows the reaction of pH indicator papers embedded with the exemplary 3CL^{pro}-specific substrate, following exposure to a pH=8.5 buffer, subsequent drying, a subsequent exposure to a solution with the 3CL^{pro} protease and a subsequent drying, upon up to 36-days of storage of the substrate-embedded pH indicator papers. As can be seen, the peptide substrate-containing paper-based platform showed long-term detection stability, indicating that it can be stored for months under unrefrigerated dry conditions without resulting in any significant change in colorimetric detection.

FIG. 9B shows the substrate-containing pH indicator paper shown in FIG. 9A, following a 36-days storage, as in FIG. 9A left images, per se and upon a subsequent exposure to a pH=8.5 buffer solution, a subsequent drying, and a subsequent exposure to distilled water instead of a protease, which serves as control. As can be seen, no color change was observed for these samples, while showing a clear colorimetric difference between the substrate-containing pH indicator paper in the absence (FIG. 9B) and the presence (FIG. 9A) of the protease, thus also indicating the long-term stability towards the detection of a protease-free sample, and illustrate the differences between the two optional results, even following a prolonged storage of the substrate-containing pH paper.

FIG. 9C shows the reaction of a substrate-free (“Clean”) paper following a 36-days storage, following exposure to a protease-containing sample and subsequently to a pH=8.5 buffer solution, and a subsequent drying. As can be seen, the addition of a protease to a clean pH paper does not result in a colorimetric change.

FIG. 9D shows the colorimetric change of two 36-days stored substrate-containing pH papers, following exposure to a pH=8.5 buffer solution and a subsequent exposure to a protease-containing sample (left) or to a protease-free sample (right). The results clearly display the change in color between a positive- and a negative-result obtained by the platform, which align with the expected results, and further demonstrate the long-term detection stability of the platform.

### Example 8

### Remarks

Overall, the novel detection methodology exemplified herein demonstrated antibody-free ultrafast and clinically-accurate detection of viral infection, while further demonstrating simplicity, long-term stability under ambient conditions and cost-effectivity, which are highly desired attributes and are required for mass screening real-world applications during a pandemic.

These detection method and device offer antibody-free pH paper-based platform, which was demonstrated for its selective POC detection of SARS-CoV-2 infection from untreated saliva samples in under less than one minute. The data obtained by this low-cost platform could easily be integrated with cellphone technology to allow at-home testing.

Implementation of this approach is made for the detection of other viral infections through the detection of specific viral proteases, including smallpox K7L protease [Aleshin et al., J Biol Chem 2012, 287 (47), 39470-39479], human rhinovirus 3CL protease [Wanga et al., Curr Protein Pept Sci 2007, 8 (1), 19-27], and poliovirus 2A protease [Castelló et al., Journal of Biomedicine and Biotechnology 2011, 2011, e369648; and Kean et al., Journal of General Virology 71 (11), 2553-2563]. Applying specific peptide substrate reaction solutions to an array of pH-paper test windows enables straightforward multiplexed diagnosis of several viral infections on a single antibody-free universal sensing platform.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of determining a presence and/or amount of a viral biomarker in a sample, the method comprising:
contacting the sample with an agent that selectively reacts with the viral biomarker and with a colorimetric indicator,
wherein said agent that selectively reacts with the viral biomarker and said colorimetric indicator are selected such that a reaction of said agent with the viral biomarker results in a colorimetric change in said colorimetric indicator, said colorimetric change being indicative of the presence and/or amount of the viral biomarker in the sample,
wherein said reaction of said agent with the viral biomarker results in a pH change and wherein said colorimetric indicator is a pH indicator.

2. The method of claim 1, wherein said viral biomarker is a proteolytic enzyme.

3. The method of claim 1, wherein said agent that selectively reacts with the viral biomarker is a peptide substrate that said viral biomarker specifically reacts with.

4. The method of claim 1, wherein the viral biomarker is a SARS-CoV-2-specific proteolytic enzyme.

5. The method of claim 1, wherein said agent that selectively reacts with said viral biomarker is a peptide substrate that said SARS-CoV-2-specific proteolytic enzyme specifically cleaves.

6. The method of claim 1, wherein said colorimetric indicator is or comprises a solid matrix and said agent is absorbed in and/or on said solid matrix.

7. The method of claim 1, wherein said contacting comprises contacting an aqueous solution that comprises said agent that selectively reacts with said viral biomarker, said colorimetric indicator, and said sample.

8. The method of claim 1, wherein said pH indicator comprises a pH paper.

9. The method of claim 8, comprising:
contacting said sample with said pH paper, to thereby absorb the sample to the pH paper; and
contacting the pH paper having the sample absorbed thereto with said aqueous solution that comprises said agent that selectively reacts with said viral biomarker,
wherein a colorimetric change in said pH paper is indicative of a presence of the viral biomarker in the sample.

10. The method of claim 8, comprising:
contacting said pH paper with said agent that selectively reacts with the viral biomarker, to thereby absorb said agent to the pH paper; and
contacting said pH paper having said agent absorbed thereto with the sample,
wherein a colorimetric change in said pH paper is indicative of a presence of the viral biomarker in the sample.

11. The method of claim 10, further comprising contacting said pH paper with an aqueous solution.

12. The method of claim 1, further comprising determining a level of said colorimetric change, said level being indicative of an amount of the viral biomarker in the sample.

13. The method of claim 12, wherein determining said level of said colorimetric change comprises converting a color that forms upon said colorimetric change to a RGB signal.

14. A kit comprising an agent that selectively reacts with a viral biomarker, and a colorimetric indicator which is such that exhibits a colorimetric change as a result of a reaction between said agent and said viral biomarker,
wherein said reaction between said agent and said viral biomarker results in a pH change.

15. The kit of claim 14, wherein said colorimetric indicator is a pH paper.

16. The kit of claim 15, wherein said agent is absorbed to said pH meter, the kit further comprising an aqueous solution.

17. The kit of claim 16, wherein said aqueous solution has a pH higher by at least 0.3 pH unit, or by at least 0.5 pH unit, or by at least one pH unit, from a pH of said solution upon said reaction between said agent and a pre-determined threshold amount of said viral biomarker in the sample.

18. A composition comprising a colorimetric indicator which is or comprises a solid matrix and an agent that selectively reacts with a viral biomarker associated with the solid matrix, wherein said reaction of said agent with said viral biomarker results in a pH change and wherein said colorimetric indicator is a pH indicator.

19. The composition of claim 18, wherein said viral biomarker is a proteolytic enzyme.

20. The composition of claim 18, wherein said agent that selectively reacts with the viral biomarker is a peptide substrate that said viral biomarker specifically reacts with.


SEQUENCE LISTING (30 sequences)

SEQ ID NO: 1 - protein, 306 aa, Severe acute respiratory syndrome-related coronavirus [sequence omitted]

SEQ ID NO: 2 - protein, 14 aa, synthetic construct
KTSAVLQSGFRKME

SEQ ID NO: 3 - protein, 110 aa, Homo sapiens
GWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 4 - protein, 306 aa, Middle East respiratory syndrome-related coronavirus
SGLVKMSHPSGDVEACMVQVTCGSMTLNGLWLDNTVWCPRHVMCPADQLSDPNYDALLISMTNHSFSVQKHIGAPANLRVVGHAMQGTLLKLTVDVANPSTPAYTFTTVKPGAAFSVLACYNGRPTGTFTVVMRPNYTIKGSFLCGSCGSVGYTKEGSVINFCYMHQMELANGTHTGSAFDGTMYGAFMDKQVHQVQLTDKYCSVNVVAWLYAAILNGCAWFVKPNRTSVVSFNEWALANQFTEFVGTQSVDMLAVKTGVAIEQLLYAIQQLYTGFQGKQILGSTMLEDEFTPEDVNMQIMGVVMQ

SEQ ID NO: 5 - protein, 306 aa, Severe acute respiratory syndrome-related coronavirus
SGFRKMAFPSGKVEGCMVQVTCGTTTLNGLWLDDTVYCPRHVICTAEDMLNPNYEDLLIRKSNHSFLVQAGNVQLRVIGHSMQNCLLRLKVDTSNPKTPKYKFVRIQPGQTFSVLACYNGSPSGVYQCAMRPNHTIKGSFLNGSCGSVGFNIDYDCVSFCYMHHMELPTGVHAGTDLEGKFYGPFVDRQTAQAAGTDTTITLNVLAWLYAAVINGDRWFLNRFTTTLNDFNLVAMKYNYEPLTQDHVDILGPLSAQTGIAVLDMCAALKELLQNGMNGRTILGSTILEDEFTPFDVVRQCSGVTFQ

SEQ ID NO: 6 - protein, 97 aa, synthetic construct
PQFSLWKRPVVTAHIEGQPVEVLLDTGADSIVAGIELGSNYSPKIVGGIGGFINTKEYKNVEIEVLNKRVRATIMTGDTPINIFGRNILASGMSLNL

SEQ ID NO: 7 - protein, 268 aa, Homo sapiens [sequence omitted]

SEQ ID NO: 8 - protein, 7 aa, synthetic construct
KTSAVLQ

SEQ ID NO: 9 - protein, 7 aa, synthetic construct
SGFRKME

SEQ ID NO: 10 - protein, 11 aa, Severe acute respiratory syndrome-related coronavirus
TSAVLQSGFRK

SEQ ID NO: 11 - protein, 11 aa, synthetic construct
SGVTFQSAVKR

SEQ ID NO: 12 - protein, 11 aa, synthetic construct
KVATVQSKMSD

SEQ ID NO: 13 - protein, 11 aa, synthetic construct
NRATLQAIASE

SEQ ID NO: 14 - protein, 11 aa, synthetic construct
SAVKLQNNELS

SEQ ID NO: 15 - protein, 11 aa, synthetic construct
ATVRLQAGNAT

SEQ ID NO: 16 - protein, 11 aa, synthetic construct
REPMLQSADAQ

SEQ ID NO: 17 - protein, 11 aa, synthetic construct
PHTVLQAVGAC

SEQ ID NO: 18 - protein, 11 aa, synthetic construct
NVATLQAENVT

SEQ ID NO: 19 - protein, 11 aa, synthetic construct
TFTRLQSLENV

SEQ ID NO: 20 - protein, 11 aa, synthetic construct
FYPKLQSSQAW

SEQ ID NO: 21 - protein, 12 aa, synthetic construct
ESATLQSGLAKS

SEQ ID NO: 22 - protein, 10 aa, synthetic construct
LAQAVRSSSR

SEQ ID NO: 23 - protein, 12 aa, synthetic construct
VNSTLQSGLRKM

SEQ ID NO: 24 - protein, 10 aa, synthetic construct
SAVLQSGFRK

SEQ ID NO: 25 - protein, 12 aa, synthetic construct
CESATLQSGLAK

SEQ ID NO: 26 - protein, 9 aa, synthetic construct
SVTLQSGYR

SEQ ID NO: 27 - protein, 11 aa, synthetic construct
SGVTFQGKFKK

SEQ ID NO: 28 - protein, 9 aa, synthetic construct
AVLQSGFKK

SEQ ID NO: 29 - protein, 11 aa, synthetic construct
REPLMQSADAS

SEQ ID NO: 30 - protein, 11 aa, synthetic construct
FYPKLQASQAW